(19) United States
(12) Patent Application Publication
(10) Pub. No.: US 2024/0177860 A1
(43) Pub. Date: May 30, 2024

(54) METHODS, SYSTEMS, AND MEDIA FOR DETERMINING A HORMONE LEVEL AND A HORMONAL STATE OF A SUBJECT

(71) Applicant: Uniq Health Ltd, Cambridge (GB)

(72) Inventors: Shardi Nahavandi, London (GB); Stephen A. Butler, Bedfordshire (GB)

(21) Appl. No.: 18/526,879

(22) Filed: Dec. 1, 2023

Related U.S. Application Data
(63) Continuation of application No. PCT/EP22/06527, filed on Jun. 3, 2022.

(30) Foreign Application Priority Data
Jun. 3, 2021 (GB) 2107973.6

(51) Int. Cl. G16H 50/20; G06T 7/00; G06V 10/774; G06V 10/82; G06V 40/16; G16B 20/00

(52) U.S. Cl. CPC G16H 50/20; G06T 7/0012; G06V 10/774; G06V 10/82; G06V 40/165; G16B 20/00; G06T 2207/20081; G06T 2207/20084; G06T 2207/30004; G06T 2207/30201

(57) ABSTRACT

Provided herein are methods, systems, and media for determining a recommended contraceptive by employing clinical decision-making tools to analyze a subject's specific biology (e.g. medical history, mental profile, hormone profile, genetic profiles, menstrual profile). Such tools may discover or analyze biological variables that can be related to side effects and their severities. The methods, systems, and media described herein allow for customized treatment/contraceptive recommends for any subject. In some embodiments, the methods, systems, and media provide information/identification of a hormonal state for a subject. Currently, contraceptive recommendations are primarily based on a one-time “snapshot” of the hormonal profile of the subject at the time the identification assay is administered to the subject. The methods, systems, and media described herein enable the identification of a more transient hormonal state of the subject.

### PRIORITY

This application claims priority to EP Patent Application No. PCT/EP2022/065273 filed Jun. 3, 2022, which is hereby incorporated by reference in its entirety and which claims the benefit of United Kingdom Patent Application No. 2107973.6, filed Jun. 3, 2021, which is hereby incorporated by reference in its entirety herein.

### BACKGROUND

Currently women's hormonal and contraceptive health is often a “one-size-fits-all” approach. Such regimens, however, ignore individualistic side effects. 1 in 2 women using hormonal contraceptives develop a side effect, and such women have an 80% higher risk of depression and a 28% higher risk of developing breast cancer. Thus, there is a need in the art to reduce both the development and the severity of side effects that result from using contraceptives.

### SUMMARY

One aspect provided herein is a computer-implemented method for determining a contraceptive recommendation for a subject, said method comprising: (a) receiving a plurality of contraceptives, wherein each of said contraceptives is associated with one or more contraceptive-specific side effects, and wherein one or more of said side effects is correlated to one or more biological variables; (b) determining a first contraceptive of the plurality of contraceptives based on one or more contraceptive-specific side effects specific to the first contraceptive; (c) transmitting a contraceptive recommendation comprising an indication of the first contraceptive; (d) receiving a subject side effect factor associated with the one or more contraceptive-specific side effects specific to the first contraceptive; and (e) updating a relationship between the first contraceptive and one or more biological variables based on the subject side effect factor, wherein the relationship indicates a likelihood of experiencing a contraceptive-specific side effect when administering the first contraceptive, and wherein updating the relationship results in increased accuracy of determining a contraceptive based on the one or more contraceptive-specific side effects. In some embodiments, each of said one or more contraceptive-specific side effects comprises a side effect severity and the first contraceptive-specific side effect comprises a first side effect severity. In some embodiments, each of said side effects having a relationship with one or more biological variables comprises each side effect severity of each side effect correlating to one or more biological variables through one or more correlation coefficients. In some embodiments, the relationship between the first contraceptive and one or more biological variables comprises the first side effect severity correlating to one or more biological variables through a first correlation coefficient. In some embodiments, updating the relationship between the first contraceptive and one or more biological variables comprises adjusting the first correlation coefficient. In some embodiments, determining a first contraceptive of the plurality of contraceptives based on one or more contraceptive-specific side effects specific to the first contraceptive comprises comparing side effect severities associated with the contraceptive-specific side effects specific to the first contraceptive and one or more contraceptive-specific side effects specific to a second contraceptive and determining the first contraceptive based on the comparing. In some embodiments, said one or more biological variables comprise a hormonal state of said subject. In some embodiments, said hormonal state comprises quantification on an androgen axis, an estrogen axis, or both. In some embodiments, said hormonal state is associated with at least one of said two or more of contraceptives. In some embodiments, said hormonal state is determined by receiving one or more images of the face of said subject, measuring one or more facial metrics based on said one or more images, generating a subject-specific score from said one or more facial metrics, receiving one or more verified hormonal states, each of said verified hormonal states associated with a verified hormonal state, determining said hormone level of said subject based on said one or more verified hormonal states and said subject-specific score, and providing an output representing said hormonal state of said subject. In some embodiments, said one or more images of said face of said subject comprises a video. In some embodiments, measuring one or more facial metrics based on said one or more images is performed by machine vision. In some embodiments, generating a subject-specific score from said one or more facial metrics is performed by a facial metric machine learning algorithm. In some embodiments, said one or more facial metrics comprise a facial width, facial height, facial coloration, mandibular width, mandibular contour, nasal width, or any combination thereof. In some embodiments, said facial metric machine learning algorithm is trained by a method comprising collecting two or more sets of said facial metrics from a database, wherein each set of said facial metrics is associated with a validated subject-specific score, creating a first training set comprising said collected set of said two or more sets of said facial metrics, training a neural network in a first stage to determine said subject-specific score using said first training set, creating a second training set comprising said first training set and said two or more sets of facial metrics whose subject-specific score was determined beyond a set threshold from said validated subject-specific score, and determining the first contraceptive based on the comparing. In some embodiments, wherein said hormonal state comprises quantification on an androgen axis, an estrogen axis, or both. In some embodiments, said hormone level is further determined based on a published scientific guideline. In some embodiments, the method further comprises receiving a measured hormonal state of said subject and feeding back said measured hormonal state to improve said hormone level determination over time. In some embodiments, the method further comprises receiving a biological variable value for one or more of the biological variables associated with said subject. In some embodiments, said one or more biological variables comprises a medical history, a mental profile, a hormone profile, a genetic profile, a menstrual profile, a lifestyle preference, a side effects, a biometric information, or any combination thereof. In some embodiments, said contraceptive machine learning algorithm is trained by a method comprising receiving two or more of said contraceptive-specific side effects from a database, wherein each of said two or more contraceptive-specific side effects is associated with said one or more of said biological variables by a verified biological correlation coefficient, creating a first training set comprising said two or more contraceptive-specific side effects, training a neural network in a first stage to determine a biological correlation coefficient, creating a second training set comprising said first training set and said contraceptive-specific side effects whose biological correlation coefficient was determined to be different than said verified biological correlation coefficient by a set value, and training said machine learning algorithm in a second stage using said second training set. In some embodiments, said contraceptive comprises a subset of contraceptives. In some embodiments, determining said contraceptive recommendation is determined based on an average of side effect severities of each of said one or more contraceptive-specific side effects, a sum of said side effect severities of each of said one or more contraceptive-specific side effects, or both. In some embodiments, said computer is a mobile device. In some embodiments, the method further comprises repeating one or more of said method steps. In some embodiments, at least a portion of said method is performed in an absence of any involvement or manual input from said subject.

One aspect provided herein is a computer-implemented method for determining a contraceptive recommendation for a subject, said method comprising: (a) receiving a set of two or more of said contraceptives, wherein each of said contraceptives is associated with one or more contraceptive-specific side effects, wherein each of said one or more contraceptive-specific side effects comprises a side effect severity, and wherein said side effect severity is correlated to one or more biological variables by one or more correlation coefficients; (b) determining said contraceptive recommendation from said set of two or more contraceptives, wherein said contraceptive recommendation is determined based on said side effect severity of each of said one or more contraceptive-specific side effects for each of said set of two or more contraceptives; (c) transmitting said contraceptive recommendation; (d) receiving a subject side effect factor from said subject after using said contraceptive recommendation; and (e) altering said one or more correlation coefficients of one or more of said contraceptive-specific side effects to improve said determination over time.

In some embodiments, said one or more biological variable values comprise a hormonal state of said subject. In some embodiments, said hormonal state comprises quantification on an androgen axis, an estrogen axis, or both. In some embodiments, said hormonal state is associated with at least one of said two or more of contraceptives. In some embodiments, said hormonal state is determined by: receiving one or more images of the face of said subject; measuring one or more facial metrics based on said one or more images; generating a subject-specific score from said one or more facial metrics; receiving one or more verified hormonal states, each of said verified hormonal states associated with a verified hormonal state; determining said hormone level of said subject based on said one or more verified hormonal states and said subject-specific score; and providing an output representing said hormonal state of said subject. In some embodiments, said one or more images of said face of said subject comprises a video. In some embodiments, (b) is performed by machine vision. In some embodiments, said one or more facial metrics comprise a facial width, facial height, facial coloration, mandibular width, mandibular contour, nasal width, or any combination thereof. In some embodiments, (b) is performed by machine vision. In some embodiments, (c) is performed by a facial metric machine learning algorithm. In some embodiments, said facial metric machine learning algorithm is trained by a method comprising: collecting two or more sets of said facial metrics from a database, wherein each set of said facial metrics is associated with a validated subject-specific score; creating a first training set comprising said collected set of said two or more sets of said facial metrics; training a neural network in a first stage to determine said subject-specific score using said first training set; creating a second training set comprising said first training set and said two or more sets of facial metrics whose subject-specific score was determined beyond a set threshold from said validated subject-specific score; and training said machine learning algorithm in a second stage using said second training set. In some embodiments, said hormone level is further determined based on a published scientific guideline. In some embodiments, the method further comprises receiving a measured hormonal state of said subject; and feeding back said measured hormonal state to improve said hormone level determination over time. In some embodiments, the method further comprises receiving a biological variable value for one or more of the biological variables associated with said subject. In some embodiments, said one or more biological variable values comprises a medical history, a mental profile, a hormone profile, a genetic profile, a menstrual profile, a lifestyle preference, a side effects, a biometric information, or any combination thereof. In some embodiments, the one or more biological variables are assessed by an online health assessment, a base hormone test, a genetic test, a mood test, a stress test, a sleep test, an energy test, a digestion test, a bowel (e.g. IBS) test, a weight test, an exercise test, or any combination thereof. In some embodiments, the online health test comprises a medical history assessment, a family medical history assessment, a mental health assessment, or any combination thereof. In some embodiments, the base hormone test comprises an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, the mood test, stress test, sleep test, energy test, or any combination thereof, comprises assessment of omega 3 levels, omega 6 levels, liver function, diabetes (HbA1c), iron levels, vitamin B9 levels, vitamin B12 levels, vitamin D levels, or any combination thereof, of the subject. In some embodiments, the digestion test, the bowel test, or both, comprise assessment of omega 3 levels, omega 6 levels, liver function, diabetes (HbA1c), insulin resistance, or any combination thereof, of the subject. In some embodiments, the weight test, the exercise test, or both, comprise assessment of liver function, iron levels, vitamin B9 levels, vitamin B12 levels, vitamin D levels, or any combination thereof, of the subject.

In some embodiments, determining said contraceptive recommendation from said set of two or more contraceptives is performed by a contraceptive machine learning algorithm. In some embodiments, said contraceptive machine learning algorithm is trained by a method comprising: receiving two or more of said contraceptive-specific side effects from a database, wherein each of said two or more contraceptive-specific side effects is associated with said one or more of said biological variables by a verified biological correlation coefficient; creating a first training set comprising said two or more contraceptive-specific side effects; training a neural network in a first stage to determine a biological correlation coefficient; creating a second training set comprising said first training set and said contraceptive-specific side effects whose biological correlation coefficient was determined to be different than said verified biological correlation coefficient by a set value; and training said machine learning algorithm in a second stage using said second training set. In some embodiments, said contraceptive comprises a subset of contraceptives. In some embodiments, determining said contraceptive recommendation is determined based on an average of side effect severities of each of said one or more contraceptive-specific side effects, a sum of said side effect severities of each of said one or more contraceptive-specific side effects, or both. In some embodiments, said computer is a mobile device. In some embodiments, the method further comprises repeating one or more of said method steps. In some embodiments, at least a portion of said method is performed in an absence of any involvement or manual input from said subject.

Another aspect provided herein is a computer-implemented system for determining a contraceptive recommendation for a subject, said system comprising: a digital processing device comprising: at least one processor, an operating system configured to perform executable instructions, a memory, and a computer program including instructions executable by the digital processing device to create an application configured to perform at least the following: (a) receiving a set of two or more of said contraceptives, wherein each of said contraceptives is associated with one or more contraceptive-specific side effects, wherein each of said one or more contraceptive-specific side effects comprises a side effect severity, and wherein said side effect severity is correlated to one or more biological variables by one or more correlation coefficients; (b) determining said contraceptive recommendation from said set of two or more contraceptives, wherein said contraceptive recommendation is determined based on said side effect severity of each of said one or more contraceptive-specific side effects for each of said set of two or more contraceptives; (c) transmitting said contraceptive recommendation; (d) receiving a subject side effect factor from said subject after using said contraceptive recommendation; and (e) altering said one or more correlation coefficients of one or more of said contraceptive-specific side effects to improve said determination over time.

In some embodiments, said one or more biological variable values comprise a hormonal state of said subject. In some embodiments, said hormonal state comprises quantification on an androgen axis, an estrogen axis, or both. In some embodiments, the hormonal state is generated from an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, said hormonal state is associated with at least one of said two or more of contraceptives. In some embodiments, said hormonal state is determined by: receiving one or more images of the face of said subject; measuring one or more facial metrics based on said one or more images; generating a subject-specific score from said one or more facial metrics; receiving one or more verified hormonal states, each of said verified hormonal states associated with a verified hormonal state; determining said hormone state of said subject based on said one or more verified hormonal states and said subject-specific score; and providing an output representing said hormonal state of said subject. In some embodiments, said one or more images of said face of said subject comprises a video. In some embodiments, (b) is performed by machine vision. In some embodiments, said one or more facial metrics comprise a facial width, facial height, facial coloration, mandibular width, mandibular contour, nasal width, or any combination thereof. In some embodiments, (b) is performed by machine vision. In some embodiments, (c) is performed by a facial metric machine learning algorithm. In some embodiments, said facial metric machine learning algorithm is trained by a system comprising: collecting two or more sets of said facial metrics from a database, wherein each set of said facial metrics is associated with a validated subject-specific score; creating a first training set comprising said collected set of said two or more sets of said facial metrics; training a neural network in a first stage to determine said subject-specific score using said first training set; creating a second training set comprising said first training set and said two or more sets of facial metrics whose subject-specific score was determined beyond a set threshold from said validated subject-specific score; and training said machine learning algorithm in a second stage using said second training set. In some embodiments, said hormone state is further determined based on a published scientific guideline. In some embodiments, said application is further to perform: receiving a measured hormonal state of said subject; and feeding back said measured hormonal state to improve said hormone state determination over time. In some embodiments, said application is further to perform receiving a biological variable value for one or more of the biological variables associated with said subject. In some embodiments, said one or more biological variable values comprises a medical history, a mental profile, a hormone profile, a genetic profile, a menstrual profile, a lifestyle preference, a side effects, a biometric information, or any combination thereof. In some embodiments, determining said contraceptive recommendation from said set of two or more contraceptives is performed by a contraceptive machine learning algorithm In some embodiments, said contraceptive machine learning algorithm is trained by a system comprising: receiving two or more of said contraceptive-specific side effects from a database, wherein each of said two or more contraceptive-specific side effects is associated with said one or more of said biological variables by a verified biological correlation coefficient; creating a first training set comprising said two or more contraceptive-specific side effects; training a neural network in a first stage to determine a biological correlation coefficient; creating a second training set comprising said first training set and said contraceptive-specific side effects whose biological correlation coefficient was determined to be different than said verified biological correlation coefficient by a set value; and training said machine learning algorithm in a second stage using said second training set. In some embodiments, said contraceptive comprises a subset of contraceptives. In some embodiments, determining said contraceptive recommendation is determined based on an average of side effect severities of each of said one or more contraceptive-specific side effects, a sum of said side effect severities of each of said one or more contraceptive-specific side effects, or both. In some embodiments, said computer is a mobile device. In some embodiments, said application is further to perform repeating one or more of said system steps. In some embodiments, at least a portion of said system is performed in an absence of any involvement or manual input from said subject.

Another aspect provided herein is a non-transitory computer-readable storage media encoded with a computer program including instructions executable by a processor to create a contraceptive recommendation application configured to perform at least the following: (a) receiving a set of two or more of said contraceptives, wherein each of said contraceptives is associated with one or more contraceptive-specific side effects, wherein each of said one or more contraceptive-specific side effects comprises a side effect severity, and wherein said side effect severity is correlated to one or more biological variables by one or more correlation coefficients; (b) determining said contraceptive recommendation from said set of two or more contraceptives, wherein said contraceptive recommendation is determined based on said side effect severity of each of said one or more contraceptive-specific side effects for each of said set of two or more contraceptives; (c) transmitting said contraceptive recommendation; (d) receiving a subject side effect factor from said subject after using said contraceptive recommendation; and (e) altering said one or more correlation coefficients of one or more of said contraceptive-specific side effects to improve said determination over time.

In some embodiments, said one or more biological variable values comprise a hormonal state of said subject. In some embodiments, said hormonal state comprises quantification on an androgen axis, an estrogen axis, or both. In some embodiments, the hormonal state is generated from an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, said hormonal state is associated with at least one of said two or more of contraceptives. In some embodiments, said hormonal state is determined by: receiving one or more images of the face of said subject; measuring one or more facial metrics based on said one or more images; generating a subject-specific score from said one or more facial metrics; receiving one or more verified hormonal states, each of said verified hormonal states associated with a verified hormonal state; determining said hormone state of said subject based on said one or more verified hormonal states and said subject-specific score; and providing an output representing said hormonal state of said subject. In some embodiments, said one or more images of said face of said subject comprises a video. In some embodiments, (b) is performed by machine vision. In some embodiments, said one or more facial metrics comprise a facial width, facial height, facial coloration, mandibular width, mandibular contour, nasal width, or any combination thereof. In some embodiments, (b) is performed by machine vision. In some embodiments, (c) is performed by a facial metric machine learning algorithm. In some embodiments, said facial metric machine learning algorithm is trained by a system comprising: collecting two or more sets of said facial metrics from a database, wherein each set of said facial metrics is associated with a validated subject-specific score; creating a first training set comprising said collected set of said two or more sets of said facial metrics; training a neural network in a first stage to determine said subject-specific score using said first training set; creating a second training set comprising said first training set and said two or more sets of facial metrics whose subject-specific score was determined beyond a set threshold from said validated subject-specific score; and training said machine learning algorithm in a second stage using said second training set. In some embodiments, said hormone state is further determined based on a published scientific guideline. In some embodiments, said application is further to perform: receiving a measured hormonal state of said subject; and feeding back said measured hormonal state to improve said hormone state determination over time. In some embodiments, said application is further to perform receiving a biological variable value for one or more of the biological variables associated with said subject. In some embodiments, said one or more biological variable values comprises a medical history, a mental profile, a hormone profile, a genetic profile, a menstrual profile, a lifestyle preference, a side effects, a biometric information, or any combination thereof. In some embodiments, determining said contraceptive recommendation from said set of two or more contraceptives is performed by a contraceptive machine learning algorithm In some embodiments, said contraceptive machine learning algorithm is trained by a system comprising: receiving two or more of said contraceptive-specific side effects from a database, wherein each of said two or more contraceptive-specific side effects is associated with said one or more of said biological variables by a verified biological correlation coefficient; creating a first training set comprising said two or more contraceptive-specific side effects; training a neural network in a first stage to determine a biological correlation coefficient; creating a second training set comprising said first training set and said contraceptive-specific side effects whose biological correlation coefficient was determined to be different than said verified biological correlation coefficient by a set value; and training said machine learning algorithm in a second stage using said second training set. In some embodiments, said contraceptive comprises a subset of contraceptives. In some embodiments, determining said contraceptive recommendation is determined based on an average of side effect severities of each of said one or more contraceptive-specific side effects, a sum of said side effect severities of each of said one or more contraceptive-specific side effects, or both. In some embodiments, said computer is a mobile device. In some embodiments, said application is further to perform repeating one or more of said system steps. In some embodiments, at least a portion of said system is performed in an absence of any involvement or manual input from said subject.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a flow chart of an exemplary method of determining contraceptive recommendation for a subject, per an embodiment herein;

FIG. 1B shows an exemplary system for determining and providing a contraceptive recommendation.

FIG. 2A shows a graphical user interface (GUI) for selecting a side effect, per an embodiment herein;

FIG. 2B shows a GUI for learning about a skin and hair side effect, per an embodiment herein;

FIG. 3A shows a GUI for a subject to enter biological information, per an embodiment herein;

FIG. 3B shows a GUI for a subject to order a test to submit a measured hormonal state, per an embodiment herein;

FIG. 4 shows a GUI for purchasing the test to submit the measured hormonal state, per an embodiment herein;

FIG. 5 shows a non-limiting example of a computing device; in this case, a device with one or more processors, memory, storage, and a network interface, per an embodiment herein;

FIG. 6 shows a non-limiting example of a web/mobile application provision system; in this case, a system providing browser-based and/or native mobile user interfaces, per an embodiment herein; and

FIG. 7 shows a non-limiting example of a cloud-based web/mobile application provision system; in this case, a system comprising an elastically load balanced, auto-scaling web server and application server resources as well synchronously replicated databases, per an embodiment herein.

FIG. 8 shows an example flow process for determining and providing a contraceptive recommendation as well as updating relationships between side effects and biological variables.

FIG. 9 shows an exemplary list of questions to be asked for determining a contraceptive recommendation.

FIG. 10 shows a matrix of contraceptives and related side effects with side effect severities for each side effect and contraceptive.

FIG. 11 shows an example user interface for displaying questions or a contraceptive recommendation to a user.

FIG. 12 shows an example improvements or deteriorations for symptoms of side effects associated with contraceptives for women who switched contraceptives based on a contraceptive recommendation.

FIG. 13 shows a user interface displaying hormone test results and explanations of relationships between hormone levels and symptoms for the user.

FIG. 14 depicts an exemplary method for providing a contraceptive recommendation.

FIG. 15 shows an example determination of facial metrics determined based on a facial image of a user who has taken a recommended contraceptive.

FIG. 16 shows an example receiver operating characteristic for determining a period of a menstrual cycle based on facial metrics determined from facial images of one or more users.

### DETAILED DESCRIPTION

Biological variables individual to each woman have a significant effect on contraceptive-related side effects, where each biological variable may or may not be affected by each women implementing a contraceptive. With more than 300 different contraceptives, each with complex multilayered side-effects that are dependent upon the specific biology of each subject (e.g. hormonal state), contraceptive selection to reduce side effects has proven to be difficult. Often, clinicians lack the knowledge and/or time to optimize treatment. treatment pathways and opt for a broadsword, one-size-fits-all, or trial-and-error approach. Such methods result in 50% of women using hormonal contraceptives developing a side effect, with those women have an 80% higher risk of depression and a 28% higher risk of developing breast cancer.

Provided herein are methods, systems, and media for determining a recommended contraceptive by employing clinical decision-making tools to analyze a subject's specific biology (e.g. medical history, mental profile, hormone profile, genetic profiles, menstrual profile). Such tools may discover or analyze biological variables that can be related to side effects and their severities.

The methods, systems, and media described herein allow for customized treatment/contraceptive recommends for any subject. In some embodiments, the methods, systems, and media provide information/identification of a hormonal state for a subject.

Currently, contraceptive recommendations are primarily based on a one-time “snapshot” of the hormonal profile of the subject at the time the identification assay is administered to the subject. The methods, systems, and media described herein enable the identification of a more transient hormonal state of the subject. This transient hormonal state provides practitioners with a more accurate hormonal profile of the subject to which contraceptive/treatment recommendations are based off. The methods, systems, and media enable contraceptive/treatment recommendations that result in significantly less side effects for subjects as compared to the current state of the art.

Further described herein are methods, systems, and media that utilize machine vision to generate a transient hormonal state of a subject based of facial parameters and changes in the facial parameters. These methods, systems, and media enable completely non-invasive and accurate diagnoses of subjects' hormonal states. This allows for the determination of contraceptive/treatment recommendations that result in significantly less side effects for subjects as compared to the current state of the art.

### Methods, Systems, and Media for Determining a Contraceptive Recommendation

Provided herein are computer-implemented methods, systems, and media for determining a contraceptive recommendation for a subject. In some embodiments, the methods, systems, and media are configured to perform: (a) receiving a plurality of contraceptives, wherein each of said contraceptives is associated with one or more contraceptive-specific side effects, and wherein one or more of said side effects is correlated to one or more biological variables; (b) determining a first contraceptive of the plurality of contraceptives based on one or more contraceptive-specific side effects specific to the first contraceptive; (c) transmitting a contraceptive recommendation comprising an indication of the first contraceptive; (d) receiving a subject side effect factor associated with the one or more contraceptive-specific side effects specific to the first contraceptive; and (e) updating a relationship between the first contraceptive and one or more biological variables based on the subject side effect factor, wherein the relationship indicates a likelihood of experiencing a contraceptive-specific side effect when administering the first contraceptive, and wherein updating the relationship results in increased accuracy of determining a contraceptive based on the one or more contraceptive-specific side effects.

Provided herein are computer-implemented methods, systems, and media for determining a contraceptive recommendation for a subject. In some embodiments, the methods, systems, and media are configured to perform: receiving a set of two or more of the contraceptives; determining the contraceptive recommendation from the set of two or more contraceptives; transmitting the contraceptive recommendation; receiving a subject side effect factor from the subject; and altering one or more correlation coefficients of one or more of contraceptive-specific side effects to improve the determination over time.

In some embodiments, the computer is a mobile device. In some embodiments, the method further comprises repeating one or more of the method steps. In some embodiments, at least a portion of the method is performed in an absence of any involvement or manual input from the subject. In some embodiments, the computer-implemented methods, systems, and media herein for determining a contraceptive recommendation for a subject employs regression analysis. In some embodiments, at least a portion of the computer-implemented methods, systems, and media herein for determining a contraceptive recommendation for a subject does not employ classification analysis.

In some embodiments, each of the contraceptives is associated with one or more contraceptive-specific side effects. In some embodiments, each of the one or more contraceptive-specific side effects comprises a side effect severity. In some embodiments, the side effect severity is correlated to one or more biological variable values. In some embodiments, the side effect severity is correlated to one or more biological variables by one or more correlation coefficients. In some embodiments, the contraceptive recommendation is determined based on the side effect severity of each of the one or more contraceptive-specific side effects. In some embodiments, the contraceptive recommendation is determined based on the side effect severity of each of the one or more contraceptive-specific side effects for each of the set of two or more contraceptives. In some embodiments, the subject side effect factor is received from the subject after implementing the contraceptive recommendation.

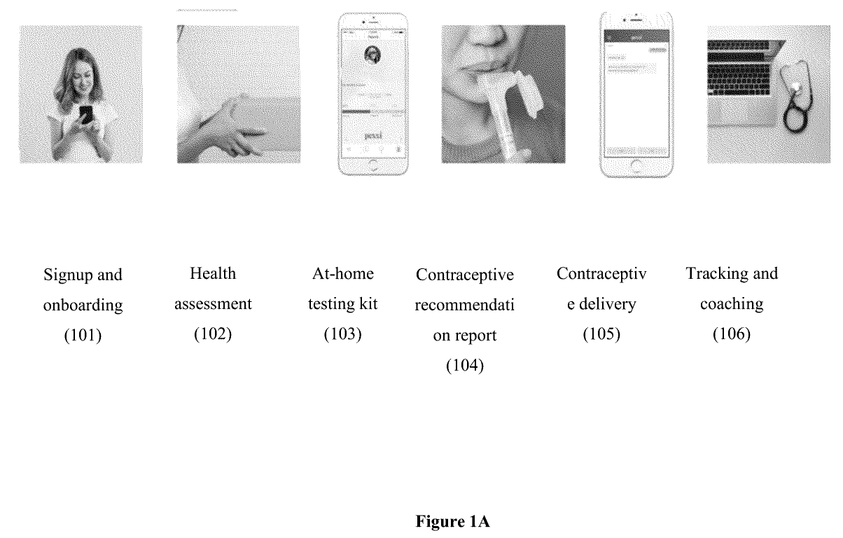

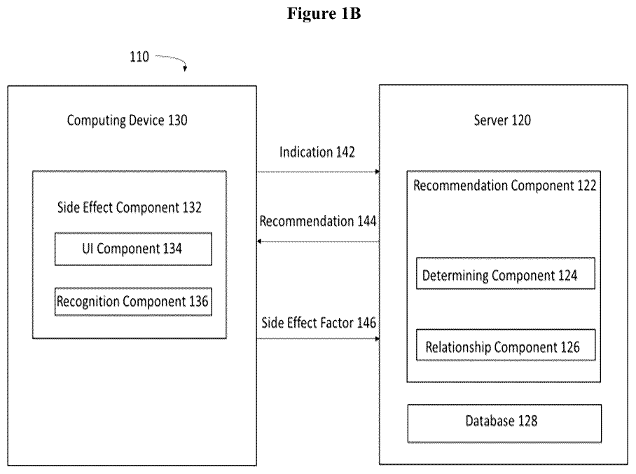

FIG. 1A shows a flow chart of an exemplary method of determining contraceptive recommendation for a subject 100. As shown, the method 100 comprises a signup and initial onboarding 101, receiving a health assessment 102, receiving a biological sample from a testing kit 103, providing the contraceptive recommendation 104, delivering the contraceptive 105, and further tracking and coaching 106.

FIG. 1B depicts an example system 110 for determining a contraceptive recommendation and updating relationships for contraceptives. In this depicted example, system 110 depicts a server 120 and a computing device 130. In this depicted example, server 120 and computing device 130 are in communication regarding one or more aspects related to contraceptives.

In this depicted embodiment, server 120 further includes recommendation component 122, and database 128. Recommendation component 122 may further include determining component 124 and relationship component 126. Further, in this depicted embodiment, computing device 130 further includes side effect component 132, UI component 134, and recognition component 136.

Recommendation component 122 may determine and provide contraceptive recommendations based on one or more actions performed by determining component 124 and relationship component 126. In this depicted example, recommendation 144 is provided by recommendation component 122 to computing device 130.

Determining component 122 of server 120 may determine one or more aspects related to contraceptives or a contraceptive recommendations. For example, determining component 122 may determine one or more contraceptives of a set of contraceptives, one or more contraceptive recommendations, one or more side effects associated with each contraceptive, one or more biological variables, one or more side effect severities, and one or more relationships between contraceptives and biological variables.

Determining component 122 may determine one or more contraceptives of a set of contraceptives. The set of contraceptives may be received by the server 122 from the computing device 130 (e.g., indicated through indication 142) or from another source, such as a network. The recommendation component 122 may determine one or more contraceptives of the set of contraceptives based on one or more side effects associated with contraceptives of the set of contraceptives or relationships between the side effects and biological variables. Side effects may include anal discharge, anemia, anger, anxiety, anxiety, appetite changes, asthenia, backache, belching, bleeding, bloating, blurred vision, bowel incontinence, breast pain/tenderness, burning or throbbing, change in bowel function, change in menstrual cycle, change in nipple position, coarse or dark hair, collapsing, comedowns, constipation, cramps, cysts, darker patches of skin, decreased breast size, deepening voice, delayed wound healing, depression, depression, diaphoresis, diarrhea, difficulty conceiving, difficulty concentrating, disturbed sleep, dizziness, dry, dark patches of skin, dysarthria, dysgeusia, dysmenorrhea, dyspareunia, dyspnea, dyspraxia, dysuria, earache, emotional changes, enlargement of the clitoris, excess hair, fatigue, feeling sick, feeling very hot or very cold, fever, flatulence, guilt, headache, heartburn, hematuria, high cholesterol levels, hirsutism, hot flushes, hungry, hypersomnia, hypertension, hypogonadism, increased muscle mass, increased thirst, indecisiveness, infertility, insomnia, irregular bleeding, irregular periods, irritability, itchiness, joint stiffness, lack of menses, lack of motivation or interest, lethargy, loss of smell, low libido, low mood, lower abdominal pain, lower abdominal pressure, menorrhagia, menstrual blood clots, menstrual hematochezia, menstrual melena, moving or speaking more slowly than usual, muscle tension or pain, nausea, neglecting hobbies and interests, new lump in the breast or armpit, nodules, oily skin/acne, pain, pallor, palpitations, papules, pelvic pain, phonophobia, photophobia, polcystic ovaries, postcoital bleeding, pressure symptoms, presyncope, primary amenorrhea, prolonged menstrual bleeding, pruritus, pustules, rectal bleeding, red or watery eyes, reduced muscle mass, regurgitation, runny or blocked nose, sadness, sebum, secondary sex characteristic development failure, seizures, shaking trembling, shiny, stretched or red skin, skin changes in the breast, skin discoloration, sneezing and coughing, social disinterest, somnolence, stomach pain, streak gonads, suicidal/self-harm thoughts, swelling, tension, thickening or swelling of the breast (or part of breast), threadlike red marks or patterns on the skin, thrush, tingling lips, tiredness and a lack of energy, trembling or shaking, unexplained aches and pains, urinary frequency, urinary tract infections, urination problems, vaginal dryness and pain, vision changes, vomiting, watery discharge, weight gain, weight loss, or any combination thereof, as described further below.

Further, determining component 122 may determine one or more side effect severities. In some embodiments, each of the one of more side effects may be associated with a side effect severity, where each side effect severity may be related to one or more biological variables through one or more relationships. Side effect severities may include a side effect severity score that may be determined by the determining component 122. The side effect severities may be determined or updated based on side effect factors associated with users. The side effect factors may be based on user input or facial images. In some embodiments, side effect severity scores may be received by the determining component 122.

Even further, determining component 122 may determine one or more biological variables associated with the one or more side effects. Biological variables may include a medical history, a mental profile, a hormone profile, a genetic profile, a menstrual profile, a lifestyle preference, a side effects, a biometric information, or any combination thereof. Determining component 122 may further determine one or more hormonal states based on one or more biological variables, as described further below. In some embodiments, the hormonal state may be specific to one or more subjects.

Relationship component 124 may create one or more relationships between one or more contraceptives and one or more biological variables based on the contraceptive-specific side effects. The relationships between side effects and biological variables may be based on how side effect severities of the side effects relate to and/or change based on biological variables. For example, a contraceptive may be associated with a contraceptive-specific side effect, which may have a specific side effect severity for a particular subject. The side effect severity may also be associated with the one or more biological variables. Relationship component 124 may create a relationship between the side effect severity associated with the contraceptive's associated side effect and the one or more biological variables. For example, in some embodiments, the relationship component may relate the side effect severity and the one or more biological variables through a correlation coefficient, as described further below.

Relationship component 124 may further update created relationships. For example, relationship component 124 may receive one or more side effect factors, such as from computing device 130. Side effect factors may include one or more components related to side effects and side effect severities, such as images of a subject or user input regarding a side effect or side effect severity. In some embodiments, the images of the subject include images of the subject's face after taking a contraceptive. The side effect factor may be associated with a contraceptive specific side effect, and the side effect severity may be updated based on the received side effect factor. A side effect severity may have an associated side effect severity score. Relationship component 124 may then update the relationship between the contraceptive and the biological variable based on the received side effect factor. For example, the updated side effect severity may cause the correlation coefficient between the side effect severity and one or more biological variables to change. Relationship component 124 then may update the relationship with the new correlation coefficient, and the updated relationship may be used when determining contraceptive recommendations.

Determining component 122 may determine one or more contraceptive recommendations based on the relationships between biological variables and the side effects and side effect severities. For example, as described above, each contraceptive in the set of contraceptives may have one or more side effects with one or more side effect severities, which are related to one or more biological variables. Determining component 122 may determine a recommendation 144 based on expected side effect severities of the side effects associated with each contraceptive in the set of contraceptives that may be based on the relationship with biological variables using on one or more techniques. For example, using a first technique, determining component 122 may determine a first contraceptive to include in the recommendation 144 because the sum of side effect severities associated with the first contraceptive are the lowest of all sums of side effect severities associated with other contraceptives in the set. As another example, using a second technique, determining component 122 may determine a second contraceptive to include in the recommendation because a specific side effect severity is below a threshold value. As yet another example, using a third technique, determining component 124 may determine a third contraceptive to include in the recommendation because a side effect severity of a specific side effect does not exist for that contraceptive (e.g., the contraceptive does not cause the side effect). In some embodiments, the determining component 124 may use received user input to determine the specific side effect. While some techniques are described above, these techniques are exemplary and other techniques may be used.

One or more machine learning models within recommendation component 122 may be utilized by determining component 124 and/or relationship component 126. Machine learning model component 128 includes one or more machine learning models. In some embodiments, relationship component 126 may utilize a first machine learning model of the one or more machine learning models, which may receive a side effect severity and a corresponding biological variable as input and output a correlation coefficient based on the side effect severity and the corresponding biological variable. The correlation coefficient may be used to update the relationship. The first machine learning model may additionally receive a side effect factor associated with the side effect severity as input, and may additionally output a new correlation coefficient based on the side effect severity, the corresponding biological variable, and the side effect factor. The first machine learning model may be used to predict side effects and side effect severities that are expected for a user when taking a contraceptive. In some embodiments, a second machine learning model of the one or more machine learning models may be utilized by determining component 124. The second machine learning model may receive one or more side effect factors, such as images of a subject face, and determine one or more facial metrics based on the one or more metrics based on the one or more side effect factors. For example, the machine learning model may receive images of the subject's face indicating one or more side effect severities, and may determine the one or more side effect severities based on the images. The side effect severities may then be used to determine one or more relationships between one or more side effect severities and a biological variable, alter one or more relationships between one or more side effect severities and a biological variable, and/or determine a contraceptive recommendation. In some embodiments, the side effect severities may be sent to the first machine learning model so that the machine learning model can determine one or more relationships between one or more side effect severities and a biological variable, alter one or more relationships between one or more side effect severities and a biological variable, and/or determine a contraceptive recommendation. Aspects and processes of the one or more machine learning are described further below.

Database 128 may store determined or related aspects of contraceptives, such as names of contraceptives, associated contraceptive side effects, side effect severities, biological variables, and correlation coefficients. In some embodiments, database 128 may further store hormonal states. In some embodiments, database 128 may store a profile for one or more subjects. A profile for a subject may include contraceptives that the subject has taken, recommended contraceptives for the subject, contraceptive-specific side effects that the subject has experienced, side effect severities that the subject has experienced, side effect factors experienced, related biological variables, and/or correlation coefficients.

In this depicted example, computing device 130 may further include side effect component 132. Side effect component 132 may further include a UI component 134 and recognition component 136 in order to capture, receive, and/or determine data related to a user and one or more contraceptives, one or more contraceptive side effects, or one or more contraceptive side effect severities.

In some embodiments, UI component 134 may include a user interface to be displayed by the computing device 130 for a user to interact with. In some embodiments, the UI component 134 may receive user input regarding one or more contraceptives, one or more contraceptive side effects, or one or more contraceptive side effect severities. In some embodiments, user input regarding one or more contraceptives may include a list of one or more contraceptives available to the user. For example, after a recommendation 144 is provided by server 120, a user associated with computing device 130 may take one or more actions regarding a recommended contraceptive (e.g., taking the recommended contraceptive). In some embodiments, the UI component 134 may display a user interface and receive user input regarding one or more side effects that the user experienced after taking the recommended contraceptive. In some embodiments, the user input may include one or more scores corresponding to the one or more experienced side effects. In other embodiments, the user input may include one or more descriptions corresponding to the one or more experienced side effects. In still other embodiments, the user input may include a general description of any number of experienced side effects. In yet another embodiment, the user input may comprise one or more images. In those embodiments, at least one of the one or more images may be of the user's face. Even further, the at least one of the one or more images may include an image of the user's face before the recommended contraceptive is taken as well as an image of the user's face after the recommended contraceptive is taken. In some embodiments, the at least one of the one or more images include an image of the user's face after the recommended contraceptive is taken but not an image of the user's face before the recommended contraceptive is taken.

Recognition component 136 may capture or receive information regarding the one or more contraceptive side effects and may further determine one or more side effect factors. In some embodiments, after the side effect factors are determined, they may be sent to server 120. For example, the recognition component 136 may include hardware such as a camera that may be used to capture one or more images. In some embodiments, the images may be the images of the user as described above. In some embodiments, the recognition component 136 may receive information regarding the one or more recommended contraceptives, the one or more contraceptive side effects, or the one or more side effect severities from UI component 134. The recognition component 136 may determine that one or more of the images, one or more aspects of the images, or one or more aspects of the information are side effect factors, such as side effect factor 146. As described above, side effect factors, such as side effect factor 146, may include one or more components related to side effects and side effect severities, such as images of a subject or user input regarding a side effect or side effect severity. In some embodiments, the images of the subject include images of the subject's face after taking a contraceptive. Side effect factor 146 may then be provided to server 120.

Thus, system 100 provides for providing recommendations of contraceptives as well as receiving information to update relationships between contraceptive side effects associated with those contraceptives and biological variables corresponding to the side effects in order to refine the relationships to improve subsequent recommendations. For example, based on the relationships between contraceptive side effects and biological variables (e.g., by correlating side effect severities and biological variables), a recommendation of one or more contraceptives may be provided. Computing device 130 may then determine one or more side effect factors through a variety of ways such as by receiving user input or capturing one or more images. The side effect factors may then be used by the server 120 to update the relationships to more accurately indicate aspects of side effects or side effect severities, which, in turn, allows for improved recommendations to be made.

### Contraceptives and Contraceptive Recommendations

In some embodiments, each of the contraceptives is associated with one or more contraceptive-specific side effects. In some embodiments, each of the one or more contraceptive-specific side effects are associated with a side effect severity. In some embodiments, each side effect may have a respective side effect severity for each user. In some embodiments, the side effect severity is correlated to one or more biological variable values. In some embodiments, the side effect severity is correlated to one or more biological variables by one or more correlation coefficients. In some embodiments, a greater correlation coefficient corresponds to a greater severity of a side effect given the subject's biological variable values.

In some embodiments, the contraceptive recommendation is determined based on the side effect severity of each of the one or more contraceptive-specific side effects. In some embodiments, the contraceptive recommendation is determined based on the side effect severity of each of the one or more contraceptive-specific side effects for each of the set of two or more contraceptives.

In some embodiments, the subject side effect factor is received from the subject after implementing the contraceptive recommendation. In some embodiments, the subject side effect factor corresponds to an actual severity of a side effect once using the recommended contraceptive. In some embodiments, the subject side effect factor is received periodically from the subject. In some embodiments, the periodicity is about 1 day, 2 days, 3 days, 5 days, 1 week, 2 weeks, 1 month, 2 months, 3 months or more, including increments therein.

In some embodiments, the contraceptive recommendation comprises a recommendation not to take a specific contraceptive if the contraceptive's side effect severity is greater than a set threshold. In some embodiments, the contraceptive recommendation comprises a recommendation not to take a specific contraceptive based on a published scientific guideline and the subject's biological variable values. In some embodiments, the contraceptive recommendation further comprises a supplement recommendation to offset one or more of the side effects.

In some embodiments, the contraceptive recommendation comprises a hormonal contraceptive recommendation, an intrauterine device recommendation, or both. In some embodiments, the hormonal contraceptive recommendation comprises an oral contraceptive, a contraceptive patch, a contraceptive ring, an injectable contraceptive, a contraceptive implant, or any combination thereof. In some embodiments, the hormonal contraceptive recommendation comprises a combined contraceptive or a progestogen only contraceptive.

### Contraceptive-Specific Side Effects

In some embodiments, each of the contraceptives is associated with one or more contraceptive-specific side effects. In some embodiments, each of the one or more contraceptive-specific side effects comprises a side effect severity. In some embodiments, the side effect severity is correlated to one or more biological variable values. In some embodiments, the side effect severity is correlated to one or more biological variables by one or more correlation coefficients. In some embodiments, the side effect severity is correlated to one or more biological variables by one or more correlation coefficients. In some embodiments, a greater correlation coefficient corresponds to a greater severity of a side effect given the subject's biological variable values.

In some embodiments, the contraceptive-specific side effect is a mental side effect. In some embodiments, the contraceptive side effect is a physical side effect. In some embodiments, the contraceptive-specific side effect is anal discharge, anemia, anger, anxiety, anxiety, appetite changes, asthenia, backache, belching, bleeding, bloating, blurred vision, bowel incontinence, breast pain/tenderness, burning or throbbing, change in bowel function, change in menstrual cycle, change in nipple position, coarse or dark hair, collapsing, comedowns, constipation, cramps, cysts, darker patches of skin, decreased breast size, deepening voice, delayed wound healing, depression, depression, diaphoresis, diarrhea, difficulty conceiving, difficulty concentrating, disturbed sleep, dizziness, dry, dark patches of skin, dysarthria, dysgeusia, dysmenorrhea, dyspareunia, dyspnea, dyspraxia, dysuria, earache, emotional changes, enlargement of the clitoris, excess hair, fatigue, feeling sick, feeling very hot or very cold, fever, flatulence, guilt, headache, heartburn, hematuria, high cholesterol levels, hirsutism, hot flushes, hungry, hypersomnia, hypertension, hypogonadism, increased muscle mass, increased thirst, indecisiveness, infertility, insomnia, irregular bleeding, irregular periods, irritability, itchiness, joint stiffness, lack of menses, lack of motivation or interest, lethargy, loss of smell, low libido, low mood, lower abdominal pain, lower abdominal pressure, menorrhagia, menstrual blood clots, menstrual hematochezia, menstrual melena, moving or speaking more slowly than usual, muscle tension or pain, nausea, neglecting hobbies and interests, new lump in the breast or armpit, nodules, oily skin/acne, pain, pallor, palpitations, papules, pelvic pain, phonophobia, photophobia, polcystic ovaries, postcoital bleeding, pressure symptoms, presyncope, primary amenorrhea, prolonged menstrual bleeding, pruritus, pustules, rectal bleeding, red or watery eyes, reduced muscle mass, regurgitation, runny or blocked nose, sadness, sebum, secondary sex characteristic development failure, seizures, shaking trembling, shiny, stretched or red skin, skin changes in the breast, skin discoloration, sneezing and coughing, social disinterest, somnolence, stomach pain, streak gonads, suicidal/self-harm thoughts, swelling, tension, thickening or swelling of the breast (or part of breast), threadlike red marks or patterns on the skin, thrush, tingling lips, tiredness and a lack of energy, trembling or shaking, unexplained aches and pains, urinary frequency, urinary tract infections, urination problems, vaginal dryness and pain, vision changes, vomiting, watery discharge, weight gain, weight loss, or any combination thereof. In some embodiments, per Table 1 below, each biological variable is associated with one or more side effects.

TABLE 1

| Biological Variable | Side Effect |
|---|---|
| Abdominal pain | Colorectal cancer |
| | Duodenal ulcers |
| | Endometrial cancer |
| | Migraine |
| | Uterine fibroids (Leiomyomas) |
| Abnormal nipple discharge | Breast cancer |
| | Amenorrhea |
| Abnormal vaginal bleeding | Endometrial cancer |
| Abnormal vaginal discharge | Cervical cancer |
| | Cervicitis |
| | Cervical cancer |
| | Endometrial cancer |
| | Thrush |
| Aching, heavy and uncomfortable legs | Varicose veins |
| Aggressive behavior | Premenstrual syndrome (PMS) |
| Alopecia | Amenorrhea |
| | Hirsutism |
| | Polycystic Ovarian Syndrome (PCOS) |
| Amenorrhea | Amenorrhea |
| | Menopause |
| | Polycystic Ovarian Syndrome (PCOS) |
| Anal discharge | Irritable bowel syndrome (IBS) |
| Anemia due to excessive menstrual bleeding | Uterine enlargement |
| Anger | Premenstrual syndrome (PMS) |
| Anxiety | Menopause |
| | Premenstrual dysphoric disorder (PMDD) |
| | Premenstrual syndrome (PMS) |
| | Hypoglycemia |
| Appetite Changes | Depression |
| | Premenstrual dysphoric disorder (PMDD) |
| | Premenstrual syndrome (PMS) |
| Asthenia | Anxiety |
| | Hypoglycemia |
| | Uterine enlargement |
| Avoiding social contact | Depression |
| Backache | Cervical cancer |
| | Irritable bowel syndrome (IBS) |
| | Uterine enlargement |
| | Uterine fibroids (Leiomyomas) |
| Belching | Dyspepsia (indigestion) |
| Bleeding (post menopause) | Cervical cancer |
| Bloating | Dyspepsia (indigestion) |
| | Irritable bowel syndrome (IBS) |
| | Premenstrual syndrome (PMS) |
| | Adenomyosis |
| | Premenstrual dysphoric disorder (PMDD) |
| | Endometriosis |
| | Ovarian cancer |
| Blurred vision | Hypoglycemia |
| | Premenstrual syndrome (PMS) |
| | Type II diabetes |
| | Hypoglycemia |
| | Premenstrual syndrome (PMS) |
| | Type II diabetes |
| Bowel incontinence | Irritable bowel syndrome (IBS) |
| Breast pain/tenderness | Breast cancer |
| | Premenstrual dysphoric disorder (PMDD) |
| | Mastalgia |
| | Breast fibrocyst |
| | Breast fibroadenoma |
| Burning or throbbing (legs) | Varicose veins |
| Change in bowel function | Colorectal cancer |
| | Irritable bowel syndrome (IBS) |
| Change in menstrual cycle | Depression |
| | Polycystic Ovarian Syndrome (PCOS) |
| | Endometriosis |
| | Adenomyosis |
| | Hypothyroidism |
| | Menorrhagia |
| Change in nipple position | Breast cancer |
| Coarse or dark hair (chest) | Hirsutism |
| | Cushing syndrome |
| | Tumour of adrenal gland/ovary |
| Coarse or dark hair (face) | Hirsutism |
| Coarse or dark hair (lower abdomen) | Hirsutism |
| Coarse or dark hair (back) | Hirsutism |
| Coarse or dark hair (inner thighs) | Hirsutism |
| Collapsing | Hypoglycemia |
| Comedones | Acne vulgaris |
| Constipation | Depression |
| | Uterine enlargement |
| | Uterine fibroids (Leiomyomas) |
| | Colorectal cancer |
| | Irritable bowel syndrome (IBS) |
| Constipation (menstrual) | Endometriosis |
| Cramps (leg muscles) | Varicose veins |
| Cramps (legs) | Uterine enlargement |
| Cramps (menstrual) | Uterine enlargement |
| Cramps (pelvic area) | Uterine enlargement |
| | Premenstrual syndrome (PMS) |
| | Polycystic Ovarian Syndrome (PCOS) |
| | Endometriosis |
| | Adenomyosis |
| Cramps (stomach) | Irritable bowel syndrome (IBS) |
| Cysts | Acne vulgaris |
| Darker patches of skin (above the upper lip) | Melasma/Chloasma (mask of pregnancy) |
| Darker patches of skin (cheeks) | Melasma/Chloasma (mask of pregnancy) |
| Darker patches of skin (forehead) | Melasma/Chloasma (mask of pregnancy) |
| Darker patches of skin (temples) | Melasma/Chloasma (mask of pregnancy) |
| Decreased breast size | Hirsutism |
| Deepening voice | Hirsutism |
| Delayed wound healing | Type II diabetes |
| Depression | Endometriosis |
| | Premenstrual dysphoric disorder (PMDD) |
| | Premenstrual syndrome (PMS) |
| Diaphoresis | Anxiety |
| | Hypoglycemia |
| | Migraine |
| Diaphoresis (night) | Menopause |
| Diarrhoea | Colorectal cancer |
| | Irritable bowel syndrome (IBS) |
| | Migraine |
| | Primary dysmenorrhea |
| Diarrhoea (menstrual) | Endometriosis |
| Difficulties in home, work or family life | Depression |
| Difficulty conceiving | Endometriosis |
| | Polycystic Ovarian Syndrome (PCOS) |
| Difficulty concentrating (brain fog) | Hypoglycemia |
| | Insulin resistance |
| | Menopause |
| | Migraine |
| | Premenstrual dysphoric disorder (PMDD) |
| | Premenstrual syndrome (PMS) |
| Disturbed sleep | Depression |
| | Premenstrual syndrome (PMS) |
| | Endometriosis |
| Dizziness | Hypoglycemia |
| | Premenstrual syndrome (PMS) |
| Dry, dark patches of skin (armpits) | Acanthosis nigricans |
| Dry, dark patches of skin (groin) | Acanthosis nigricans |
| Dry, dark patches of skin (neck) | Acanthosis nigricans |
| Dry, itchy and thin skin (over affected vein) | Varicose veins |
| Dysarthria | Hypoglycemia |
| Dyschezia (during menstruation) | Endometriosis |
| Dysgeusia | Dyspepsia (indigestion) |
| Dysmenorrhea | Uterine fibroids (Leiomyomas) |
| | Adenomyosis |
| | Menorrhagia |
| Dyspareunia | Cervical cancer |
| | Cervicitis |
| | Endometrial cancer |
| | Endometriosis |
| | Menopause |
| | Thrush |
| | Uterine enlargement |
| | Uterine fibroids (Leiomyomas) |
| Dyspnea | Anxiety |
| | Menorrhagia |
| | Pregnancy |
| Dyspraxia | Hypoglycemia |
| Dysuria | Cervicitis |
| | Cystitis |
| | Endometrial cancer |
| Dysuria (during menstruation) | Endometriosis |
| Earache | Hayfever |
| Emotional changes | Hypoglycemia |
| | Premenstrual syndrome (PMS) |
| | Menopause |
| | Perimenopause |
| | Puberty |
| | Pregnancy |
| Enlargement of the clitoris | Hirsutism |
| Excess hair (back) | Hirsutism |
| Excess hair (chest) | Hirsutism |
| Excess hair (face) | Amenorrhea |
| | Hirsutism |
| Excess hair (inner thighs) | Hirsutism |
| Excess hair (lower abdomen) | Hirsutism |
| Fatigue | Anxiety |
| | Colorectal cancer |
| | Cystitis |
| | Hypoglycemia |
| | Menorrhagia |
| | Premenstrual dysphoric disorder (PMDD) |
| | Premenstrual syndrome (PMS) |
| | Primary dysmenorrhea |
| | Type II diabetes |
| Feeling anxious or worried | Depression |
| Feeling guilt-ridden | Depression |
| Feeling hungry | Hypoglycemia |
| Feeling hungry | Insulin resistance |
| Feeling of despair | Anxiety |
| Feeling of helplessness | Depression |
| Feeling of hopelessness | Anxiety |
| | Depression |
| Feeling of sadness | Anxiety |
| Feeling of tension | Anxiety |
| Feeling shaky or trembling | Hypoglycemia |
| Feeling sick (during menstruation) | Endometriosis |
| Feeling sick (nausea) | Dyspepsia (indigestion) |
| | Irritable bowel syndrome (IBS) |
| Feeling tearful | Depression |
| Feeling very hot or very cold | Migraine |
| Fever | Primary dysmenorrhea |
| Flatulence | Dyspepsia (indigestion) |
| | Irritable bowel syndrome (IBS) |
| Haematuria | Cystitis |
| Headache | Amenorrhea |
| | Anxiety |
| | Hayfever |
| | Menopause |
| | Premenstrual dysphoric disorder (PMDD) |
| | Premenstrual syndrome (PMS) |
| | Primary dysmenorrhea |
| Heartburn | Dyspepsia (indigestion) |
| High cholesterol levels | Insulin resistance |
| Hirsutism | Polycystic Ovarian Syndrome (PCOS) |
| Hot flushes | Menopause |
| Hypersomnia | Premenstrual dysphoric disorder (PMDD) |
| Hypertension | Insulin resistance |
| Hypogonadism | Ovarian Dysgenesis |
| Increased muscle mass | Hirsutism |
| Increased thirst | Type II diabetes |
| Indecisiveness | Depression |
| Infertility | Ovarian Dysgenesis |
| | Polycystic Ovarian Syndrome (PCOS) |
| | Uterine fibroids (Leiomyomas) |
| Insomnia | Anxiety |
| | Menopause |
| | Premenstrual dysphoric disorder (PMDD) |
| Irregular bleeding (between menstrual periods) | Cervicitis |
| | Uterine polyps |
| | Cervical ectropian |
| | Cervical cancer |
| Irregular periods | Menopause |
| | Polycystic Ovarian Syndrome (PCOS) |
| Irritability | Anxiety |
| | Depression |
| | Hypoglycemia |
| | Hypoglycemia |
| | Premenstrual dysphoric disorder (PMDD) |
| | Premenstrual syndrome (PMS) |
| Itchy (ears) | Hayfever |
| Itchy (eyes) | Hayfever |
| Itchy (mouth) | Hayfever |
| Itchy (nose) | Hayfever |
| Itchy (skin) | Acanthosis nigricans |
| Itchy (skin) | Telangiectasia (Spider Veins) |
| Itchy (throat) | Hayfever |
| Joint stiffness | Menopause |
| | Oedema |
| Lack of menses | Amenorrhea |
| Lack of motivation or interest | Depression |
| Lethargy | Depression |
| | Insulin resistance |
| | Premenstrual syndrome (PMS) |
| Loss of smell | Hayfever |
| Low libido | Depression |
| | Menopause |
| Low mood | Depression |
| | Menopause |
| Low self-esteem | Depression |
| Lower abdominal pain | Cystitis |
| | Menorrhagia |
| Lower abdominal pressure | Adenomyosis |
| | Ovarian cyst |
| | Uterine fibroids (Leiomyomas) |
| | Ovarian cancer |
| | Endometrial cancer |
| Menorrhagia | Adenomyosis |
| | Endometriosis |
| | Uterine enlargement |
| | Uterine fibroids (Leiomyomas) |
| Menstrual blood clots | Menorrhagia |
| | Fibroids |
| Menstrual hematochezia | Endometriosis |
| Menstrual melena | Endometriosis |
| Menstrual pain | Primary dysmenorrhea |
| | Polycystic Ovarian Syndrome (PCOS) |
| | Endometriosis |
| | Adenomyosis |
| | Ovarian cyst |
| Moderate or severe throbbing pain | Migraine |
| (head/neck/face) | |
| Moving or speaking more slowly than usual | Depression |
| Muscle tension or pain | Anxiety |
| Nausea | Migraine |
| | Primary dysmenorrhea |
| | Pregnancy |
| Neglecting hobbies and interests | Depression |
| New lump in the breast or armpit | Breast cancer |
| | Fibroadenoma of breast |
| | Breast cyst |
| Nodules | Acne vulgaris |
| Oily skin/acne | Amenorrhea |
| | Hirsutism |
| | Polycystic Ovarian Syndrome (PCOS) |
| Pain (related to pressure on venules) | Telangiectasia (Spider Veins) |
| Pain (temples/forehead | Hayfever |
| Pallor | Hypoglycemia |
| Palpitations | Hypoglycemia |
| | Menopause |
| | Anxiety |
| Papules | Acne vulgaris |
| Pelvic pain | Amenorrhea |
| | Cervical cancer |
| | Endometriosis |
| | Ovarian cysts |
| | Ovarian cancer |
| | Uterine fibroids (Leiomyomas) |
| Phonophobia | Migraine |
| Photophobia | Migraine |
| Polcystic ovaries | Polycystic Ovarian Syndrome (PCOS) |
| Postcoital bleeding | Cervicitis |
| | Cervical cancer |
| Pressure symptoms (uterus and surrounding | Uterine enlargement |
| structures) | |
| Presyncope | Primary dysmenorrhea |
| Primary Amenorrhea | Ovarian Dysgenesis |
| Prolonged menstrual bleeding | Menorrhagia |
| Pruritus | Menopause |
| | Thrush |
| | Type II diabetes |
| Pustules | Acne vulgaris |
| Rectal bleeding | Colorectal cancer |
| Recurrent urinary tract infections (UTIs) | Menopause |
| Red or watery eyes | Hayfever |
| Reduced muscle mass | Menopause |
| Regurgitation | Dyspepsia (indigestion) |
| Runny or blocked nose | Hayfever |
| Sebum | Acne vulgaris |
| Secondary Sex Characteristic Development | Ovarian Dysgenesis |
| Failure | |
| Seizures | Hypoglycemia |
| Shiny, stretched or red skin | Oedema |
| Skin changes in the breast (puckering, dimpling, | Breast cancer |
| rash, redness) | |
| Skin discolouration | Oedema |
| Sneezing and coughing | Hayfever |
| Social Disinterest | Premenstrual dysphoric disorder (PMDD) |
| Somnolence | Hypoglycemia |
| Stomach pain | Irritable bowel syndrome (IBS) |
| Stomach pain, nausea, or digestive trouble | Anxiety |
| Streak Gonads | Ovarian Dysgenesis |
| Suicidal/self harm thoughts | Depression |
| Swelling (ankles, feet or legs) | Oedema |
| Swelling (feet and ankles) | Varicose veins |
| Swelling (legs) | Uterine enlargement |
| Swollen, enlarged blue/dark purple veins | Varicose veins |
| (legs/feet) | |
| Tearful | Hypoglycemia |
| Thickening or swelling of the breast (or part of | Breast cancer |
| breast) | |
| Threadlike red marks or patterns on the skin | Telangiectasia (Spider Veins) |
| Thrush | Type II diabetes |
| Tingling lips | Hypoglycemia |
| Tiredness and a lack of energy | Irritable bowel syndrome (IBS) |
| Trembling or shaking | Anxiety |
| Unexplained aches and pains | Depression |
| Unusual bleeding (post menopause) | Uterine enlargement |
| Urinary frequency | Cervicitis |
| | Cystitis |
| | Uterine enlargement |
| | Uterine fibroids (Leiomyomas) |
| Urinary frequency (night) | Type II diabetes |
| Urination problems | Irritable bowel syndrome (IBS) |
| Vaginal dryness and pain | Menopause |
| Vision changes | Amenorrhea |
| Vomiting | Duodenal ulcers |
| | Migraine |
| | Primary dysmenorrhea |
| Watery discharge | Uterine enlargement |
| Weight gain | Polycystic Ovarian Syndrome (PCOS) |
| | Premenstrual dysphoric disorder (PMDD) |
| | Uterine enlargement |
| | Insulin resistance |
| Weight loss | Colorectal cancer |
| | Duodenal ulcers |
| | Endometrial cancer |
| | Type II diabetes |

### Hormonal States and Biological Variables

In some embodiments, the methods and systems described herein receives a biological variable value for one or more of the biological variables associated with the subject. In some embodiments, the one or more biological variable values comprises a medical history, a mental profile, a hormone profile, a genetic profile, a menstrual profile, a lifestyle preference, a side effects, a biometric information, or any combination thereof. In some embodiments, the biological variable value of the subject is received periodically from the subject. In some embodiments, the periodicity is about 1 day, 2 days, 3 days, 5 days, 1 week, 2 weeks, 1 month, 2 months, 3 months or more, including increments therein. In some embodiments, these biological variables are utilized to determine the hormonal state of the subject.

In some embodiments, the hormonal state is determined by: (a) receiving one or more biological variables associated with the subject; (b) generating a subject-specific score from the one or more biological variables associated with the subject; (d) receiving one or more verified hormonal states, each of the verified hormonal states associated with a verified hormonal state; (e) determining the hormone level of the subject based on the one or more verified hormonal states and the subject-specific score; and (f) providing an output representing the hormonal state of the subject. In some embodiments, the one or more biological variable values comprises a medical history, a mental profile, a hormone profile, a genetic profile, a menstrual profile, a lifestyle preference, a side effects, a biometric information, or any combination thereof. In some embodiments, the subject side effect factor is received from the subject after implementing the contraceptive recommendation. In some embodiments, the biological variable value of the subject is received periodically from the subject. In some embodiments, the periodicity is about 1 day, 2 days, 3 days, 5 days, 1 week, 2 weeks, 1 month, 2 months, 3 months or more, including increments therein. In some embodiments, these biological variables are utilized to determine the hormonal state of the subject. In some embodiments, the one or more biological variables are assessed by an online health assessment, a base hormone test, a genetic test, a mood test, a stress test, a sleep test, an energy test, a digestion test, a bowel (e.g. IBS) test, a weight test, an exercise test, or any combination thereof. In some embodiments, the online health test comprises a medical history assessment, a family medical history assessment, a mental health assessment, or any combination thereof. In some embodiments, the base hormone test comprises an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, the mood test, stress test, sleep test, energy test, or any combination thereof, comprises assessment of omega 3 levels, omega 6 levels, liver function, diabetes (HbA1c), iron levels, vitamin B9 levels, vitamin B12 levels, vitamin D levels, or any combination thereof, of the subject. In some embodiments, the digestion test, the bowel test, or both, comprise assessment of omega 3 levels, omega 6 levels, liver function, diabetes (HbA1c), insulin resistance, or any combination thereof, of the subject. In some embodiments, the weight test, the exercise test, or both, comprise assessment of liver function, iron levels, vitamin B9 levels, vitamin B12 levels, vitamin D levels, or any combination thereof, of the subject. In some embodiments, the hormonal state is generated from an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject.

In some embodiments, the one or more biological variables are assessed by an online health assessment, a base hormone test, a genetic test, a mood test, a stress test, a sleep test, an energy test, a digestion test, a bowel (e.g. IBS) test, a weight test, an exercise test, or any combination thereof. In some embodiments, the online health test comprises a medical history assessment, a family medical history assessment, a mental health assessment, or any combination thereof. In some embodiments, the base hormone test comprises an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, the mood test, stress test, sleep test, energy test, or any combination thereof, comprises assessment of omega 3 levels, omega 6 levels, liver function, diabetes (HbA1c), iron levels, vitamin B9 levels, vitamin B12 levels, vitamin D levels, or any combination thereof, of the subject. In some embodiments, the digestion test, the bowel test, or both, comprise assessment of omega 3 levels, omega 6 levels, liver function, diabetes (HbA1c), insulin resistance, or any combination thereof, of the subject. In some embodiments, the weight test, the exercise test, or both, comprise assessment of liver function, iron levels, vitamin B9 levels, vitamin B12 levels, vitamin D levels, or any combination thereof, of the subject.

In some embodiments, the hormonal state is acanthosis nigricans, acne vulgaris, adenomyosis, amenorrhea, anxiety, breast cancer, breast cyst, breast fibroadenoma, breast fibrocyst, cervical cancer, cervical ectropian, cervicitis, colorectal cancer, cushing syndrome, cystitis, depression, duodenal ulcers, dyspepsia, endometrial cancer, endometriosis, fibroadenoma of breast, fibroids, hayfever, hirsutism, hypoglycemia, hypothyroidism, insulin resistance, irritable bowel syndrome, mastalgia, melasma/chloasma, menopause, menorrhagia, migraine, oedema, ovarian cancer, ovarian cyst, ovarian cysts, ovarian dysgenesis, perimenopause, polycystic ovarian syndrome, pregnancy, premenstrual dysphoric disorder, premenstrual syndrome, primary dysmenorrhea, puberty, telangiectasia, thrush, tumour of adrenal gland/ovary, type ii diabetes, uterine enlargement, uterine fibroids, uterine polyps, varicose veins, or any combination thereof.

In some embodiments, the hormonal state is generated from an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject.

In some embodiments, the one or more biological variables are assessed by an online health assessment, a base hormone test, a genetic test, a mood test, a stress test, a sleep test, an energy test, a digestion test, a bowel (e.g. IBS) test, a weight test, an exercise test, or any combination thereof. In some embodiments, the online health test comprises a medical history assessment, a family medical history assessment, a mental health assessment, or any combination thereof. In some embodiments, the base hormone test comprises an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, the mood test, stress test, sleep test, energy test, or any combination thereof, comprises assessment of omega 3 levels, omega 6 levels, liver function, diabetes (HbA1c), iron levels, vitamin B9 levels, vitamin B12 levels, vitamin D levels, or any combination thereof, of the subject. In some embodiments, the digestion test, the bowel test, or both, comprise assessment of omega 3 levels, omega 6 levels, liver function, diabetes (HbA1c), insulin resistance, or any combination thereof, of the subject. In some embodiments, the weight test, the exercise test, or both, comprise assessment of liver function, iron levels, vitamin B9 levels, vitamin B12 levels, vitamin D levels, or any combination thereof, of the subject.

In some embodiments, the side effect severity is correlated to one or more biological variable values. In some embodiments, the side effect severity is correlated to one or more biological variables by one or more correlation coefficients. In some embodiments, determining the contraceptive recommendation from the set of two or more contraceptives is performed by a contraceptive machine learning algorithm. In some embodiments, the contraceptive machine learning algorithm is trained by a method comprising: receiving two or more of the contraceptive-specific side effects from a database, wherein each of the two or more contraceptive-specific side effects is associated with the one or more of the biological variable values by a verified biological correlation coefficient; creating a first training set comprising the two or more contraceptive-specific side effects; training a neural network in a first stage to determine a biological correlation coefficient; creating a second training set comprising the first training set and the contraceptive-specific side effects whose biological correlation coefficient was determined to be different than the verified biological correlation coefficient by a set value; and training the machine learning algorithm in a second stage using the second training set. In some embodiments, the contraceptive comprises a subset of contraceptives. In some embodiments, determining the contraceptive recommendation is determined based on an average of side effect severities of each of the one or more contraceptive-specific side effects, a sum of the side effect severities of each of the one or more contraceptive-specific side effects, or both.

In some embodiments, the biological variable is a subject's medical history, mental profile, hormone profile, genetic profile, menstrual profile, or any combination thereof. In some embodiments, the one or more biological variables are assessed by an online health assessment, a base hormone test, a genetic test, a mood test, a stress test, a sleep test, an energy test, a digestion test, a bowel (e.g. IBS) test, a weight test, an exercise test, or any combination thereof. In some embodiments, the online health test comprises a medical history assessment, a family medical history assessment, a mental health assessment, or any combination thereof. In some embodiments, the base hormone test comprises an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, the mood test, stress test, sleep test, energy test, or any combination thereof, comprises assessment of omega 3 levels, omega 6 levels, liver function, diabetes (HbA1c), iron levels, vitamin B9 levels, vitamin B12 levels, vitamin D levels, or any combination thereof, of the subject. In some embodiments, the digestion test, the bowel test, or both, comprise assessment of omega 3 levels, omega 6 levels, liver function, diabetes (HbA1c), insulin resistance, or any combination thereof, of the subject. In some embodiments, the weight test, the exercise test, or both, comprise assessment of liver function, iron levels, vitamin B9 levels, vitamin B12 levels, vitamin D levels, or any combination thereof, of the subject.

In some embodiments, the biological variable is based at least in part on the subject's age, weight, height, ethnicity, genome, allergies, medical regimen, disease, condition, blood pressure, body fat percentage, anal discharge, anemia, anger, anxiety, anxiety, appetite changes, asthenia, backache, belching, bleeding, bloating, blurred vision, bowel incontinence, breast pain/tenderness, burning or throbbing, change in bowel function, change in menstrual cycle, change in nipple position, coarse or dark hair, collapsing, comedowns, constipation, cramps, cysts, darker patches of skin, decreased breast size, deepening voice, delayed wound healing, depression, depression, diaphoresis, diarrhea, difficulty conceiving, difficulty concentrating, disturbed sleep, dizziness, dry, dark patches of skin, dysarthria, dysgeusia, dysmenorrhea, dyspareunia, dyspnea, dyspraxia, dysuria, earache, emotional changes, enlargement of the clitoris, excess hair, fatigue, feeling sick, feeling very hot or very cold, fever, flatulence, guilt, headache, heartburn, hematuria, high cholesterol levels, hirsutism, hot flushes, hungry, hypersomnia, hypertension, hypogonadism, increased muscle mass, increased thirst, indecisiveness, infertility, insomnia, irregular bleeding, irregular periods, irritability, itchiness, joint stiffness, lack of menses, lack of motivation or interest, lethargy, loss of smell, low libido, low mood, lower abdominal pain, lower abdominal pressure, menorrhagia, menstrual blood clots, menstrual hematochezia, menstrual melena, moving or speaking more slowly than usual, muscle tension or pain, nausea, neglecting hobbies and interests, new lump in the breast or armpit, nodules, oily skin/acne, pain, pallor, palpitations, papules, pelvic pain, phonophobia, photophobia, polcystic ovaries, postcoital bleeding, pressure symptoms, presyncope, primary amenorrhea, prolonged menstrual bleeding, pruritus, pustules, rectal bleeding, red or watery eyes, reduced muscle mass, regurgitation, runny or blocked nose, sadness, sebum, secondary sex characteristic development failure, seizures, shaking trembling, shiny, stretched or red skin, skin changes in the breast, skin discoloration, sneezing and coughing, social disinterest, somnolence, stomach pain, streak gonads, suicidal/self-harm thoughts, swelling, tension, thickening or swelling of the breast (or part of breast), threadlike red marks or patterns on the skin, thrush, tingling lips, tiredness and a lack of energy, trembling or shaking, unexplained aches and pains, urinary frequency, urinary tract infections, urination problems, vaginal dryness and pain, vision changes, vomiting, watery discharge, weight gain, weight loss, or any combination thereof. In some embodiments, the biological variable value is determined from sample received from the subject.

In some embodiments, the hormonal state is a transient hormonal state. In some embodiments, the hormonal state is an instant hormonal sate. In some embodiments, the hormonal state is an accumulated hormonal state. In some embodiments, the one or more biological variable values comprise a hormonal state of the subject. In some embodiments, the hormonal state comprises quantification on an androgen axis, an estrogen axis, or both. In some embodiments, the hormonal state is generated from an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject. In some embodiments, the hormonal state is associated with at least one of the two or more of contraceptives.

In some embodiments, the method further comprises receiving a measured hormonal state of the subject; and feeding back the measured hormonal state to improve the hormone level determination over time. In some embodiments, the subject side effect factor is received from the subject after implementing the contraceptive recommendation. In some embodiments, the measured hormonal state of the subject is received periodically from the subject. In some embodiments, the periodicity is about 1 day, 2 days, 3 days, 5 days, 1 week, 2 weeks, 1 month, 2 months, 3 months or more, including increments therein. In some embodiments, the hormonal state of the subject is measured from a biological sample received from the subject.

### Facial Metrics

Daily facial metrics have been associated with women's' hormone states. In one example, high facial width-to-height ratio has been associated with higher testosterone levels. In another example, a women's facial skin becomes redder when her estradiol is high. As such, the methods, systems, and media herein employ 3D facial visualization and analysis software to determine a women's facial metrics and hormonal states

In some embodiments, the hormonal state is a transient hormonal state. In some embodiments, the hormonal state is an instant hormonal state. In some embodiments, the hormonal state is an accumulated hormonal state. In some embodiments, the one or more biological variable values comprise a hormonal state of the subject. In some embodiments, the hormonal state comprises quantification on an androgen axis, an estrogen axis, or both. In some embodiments, the hormonal state is generated from an assessment of oestradiol levels, thyroid levels, luteinizing hormone levels, follicle-stimulating hormone levels, free androgen index, sex hormone binding globulin levels, testosterone levels, progesterone levels, androstenedione levels, dehydroepiandrosterone levels, prolactin levels, or any combination thereof, of the subject based on the facial metrics and/or changes thereof. In some embodiments, the hormonal state is associated with at least one of the two or more of contraceptives.

In some embodiments, the hormonal state is determined by: (a) receiving one or more images of the face of the subject; (b) measuring one or more facial metrics based on the one or more images; (c) generating a subject-specific score from the one or more facial metrics; (d) receiving one or more verified hormonal states, each of the verified hormonal states associated with a verified hormonal state; (e) determining the hormone level of the subject based on the one or more verified hormonal states and the subject-specific score; and (f) providing an output representing the hormonal state of the subject. In some embodiments, the one or more images of the face of the subject comprises a video. In some embodiments, (b) is performed by machine vision. In some embodiments, the one or more facial metrics comprise a facial width, facial height, facial coloration, mandibular width, mandibular contour, nasal width, or any combination thereof. In some embodiments, the subject side effect factor is received from the subject after implementing the contraceptive recommendation. In some embodiments, the one or more images of the face of the subject is received periodically from the subject. In some embodiments, the periodicity is about 1 day, 2 days, 3 days, 5 days, 1 week, 2 weeks, 1 month, 2 months, 3 months or more, including increments therein.

In some embodiments, step (c) is performed by a facial metric machine learning algorithm. In some embodiments, the facial metric machine learning algorithm is trained by a method comprising: collecting two or more sets of the facial metrics from a database, wherein each set of the facial metrics is associated with a validated subject-specific score; creating a first training set comprising the collected set of the two or more sets of the facial metrics; training a neural network in a first stage to determine the subject-specific score using the first training set; creating a second training set comprising the first training set and the two or more sets of facial metrics whose subject-specific score was determined beyond a set threshold from the validated subject-specific score; and training the machine learning algorithm in a second stage using the second training set. In some embodiments, the hormone level is further determined based on a published scientific guideline.

### Machine Learning

In some embodiments, a contraceptive machine learning algorithm is utilized to determine the contraceptive recommendation from the set of two or more contraceptives. In some embodiments, the contraceptive machine learning algorithm is trained by a method comprising: receiving two or more of said contraceptive-specific side effects from a database, wherein each of said two or more contraceptive-specific side effects is associated with said one or more of said biological variables by a verified biological correlation coefficient; creating a first training set comprising said two or more contraceptive-specific side effects; training a neural network in a first stage to determine a biological correlation coefficient; creating a second training set comprising said first training set and said contraceptive-specific side effects whose biological correlation coefficient was determined to be different than said verified biological correlation coefficient by a set value; and training said machine learning algorithm in a second stage using said second training set.

In some embodiments, a facial metric machine learning algorithm is utilized to measure one or more facial metrics based on the one or more images. In some embodiments, the facial metric machine learning algorithm is trained by a method comprising: collecting two or more sets of the facial metrics from a database, wherein each set of the facial metrics is associated with a validated subject-specific score; creating a first training set comprising the collected set of the two or more sets of the facial metrics; training a neural network in a first stage to determine the subject-specific score using the first training set; creating a second training set comprising the first training set and the two or more sets of facial metrics whose subject-specific score was determined beyond a set threshold from the validated subject-specific score; and training the machine learning algorithm in a second stage using the second training set.

In some embodiments, the machine learning algorithms herein employ one or more forms of labels including but not limited to human annotated labels and semi-supervised labels. In some embodiments, the machine learning algorithm utilizes regression modeling, wherein relationships between predictor variables and dependent variables are determined and weighted. In one embodiment, for example, the facial metric is a dependent variable and is derived from the one or more images of the face of said subject. In another embodiment, for example, the contraceptive recommendation is a dependent variable and is derived from the contraceptive-specific side effects.

The human annotated labels can be provided by a hand-crafted heuristic. For example, the hand-crafted heuristic can comprise examining differences between facial and non-facial features. The semi-supervised labels can be determined using a clustering technique to find properties similar to those flagged by previous human annotated labels and previous semi-supervised labels. The semi-supervised labels can employ a XGBoost, a neural network, or both.

In some embodiments, the machine learning algorithms herein employ distant supervision. The distant supervision method can create a large training set seeded by a small hand-annotated training set. The distant supervision method can comprise positive-unlabeled learning with the training set as the ‘positive’ class. The distant supervision method can employ a logistic regression model, a recurrent neural network, or both. The recurrent neural network can be advantageous for Natural Language Processing (NLP) machine learning.

Examples of machine learning algorithms can include a support vector machine (SVM), a naïve Bayes classification, a random forest, a neural network, deep learning, or other supervised learning algorithm or unsupervised learning algorithm for classification and regression. The machine learning algorithms can be trained using one or more training datasets.

In some embodiments, a machine learning algorithm is used to select catalogue images and recommend project scope. A non-limiting example of a multi-variate linear regression model algorithm is seen below: probability=A₀+A₁(X₁)+A₂(X₂)+A₃(X₃)+A₄(X₄)+A₅(X₅)+A₆(X₆)+A₇(X₇) . . . wherein Aᵢ (A₁, A₂, A₃, A₄, A₅, A₆, A₇, . . . ) are “weights” or coefficients found during the regression modeling; and Xᵢ (X₁, X₂, X₃, X₄, X₅, X₆, X₇, . . . ) are data collected from the User. Any number of Aᵢ and Xᵢ variable can be included in the model. For example, in a non-limiting example wherein there are 7 Xᵢ terms, X₁ is the number of contraceptive-specific side effects, X₂ is the number of biological variables, and X₃ is the biological correlation coefficient. In some embodiments, the programming language “R” is used to run the model.

### Graphical User Interfaces

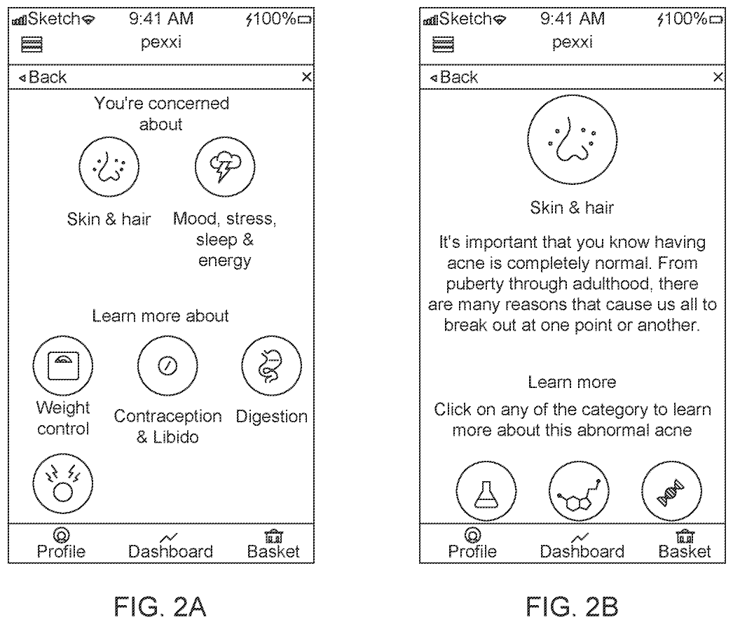

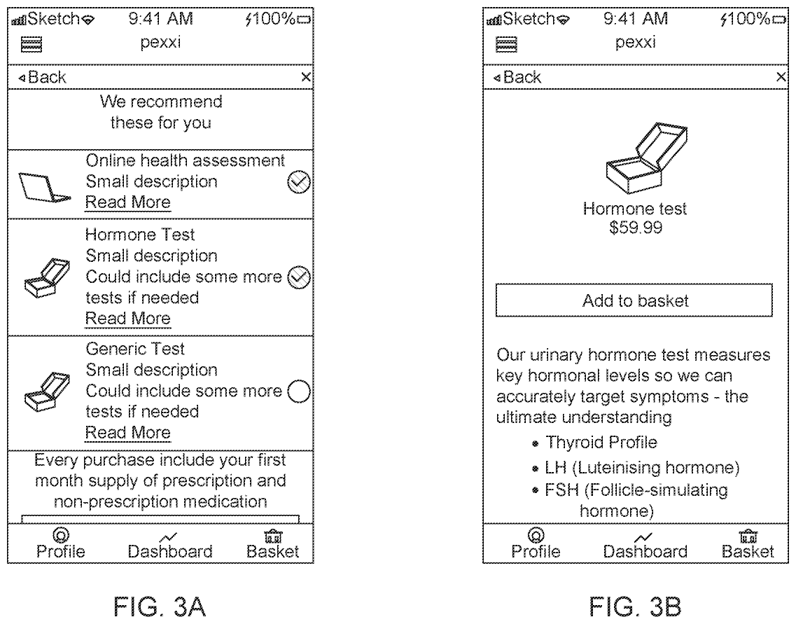

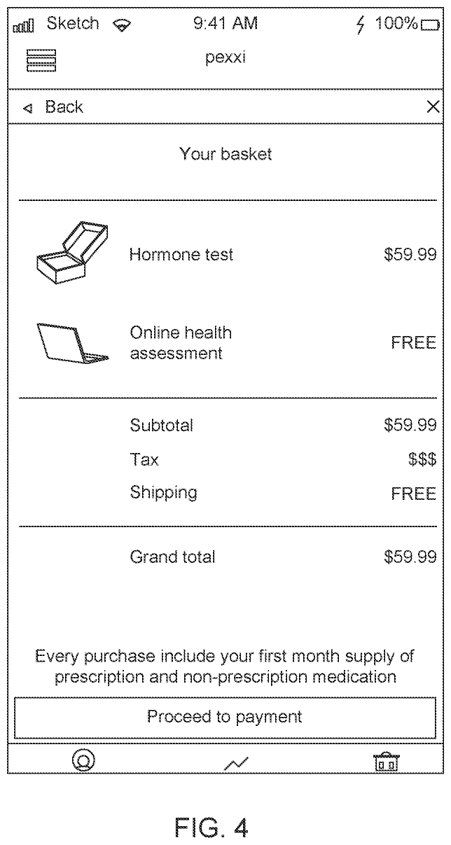

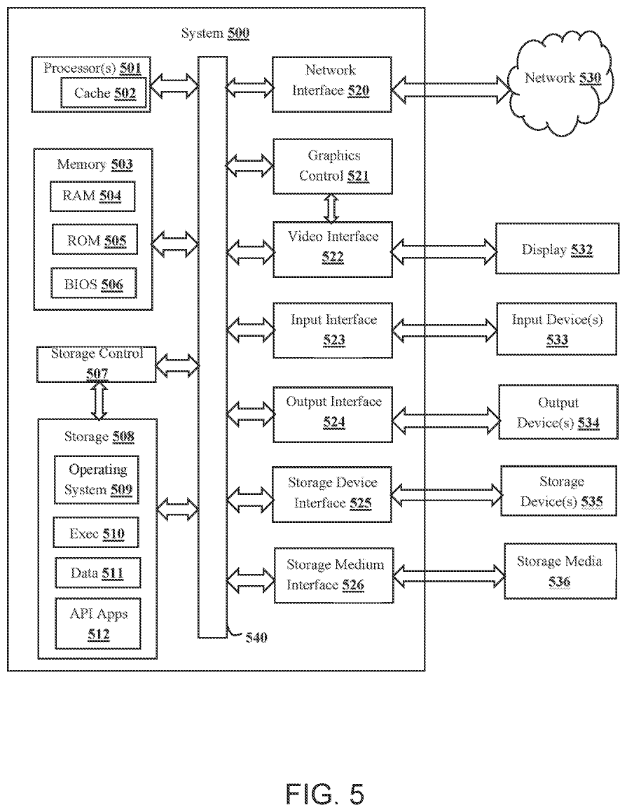

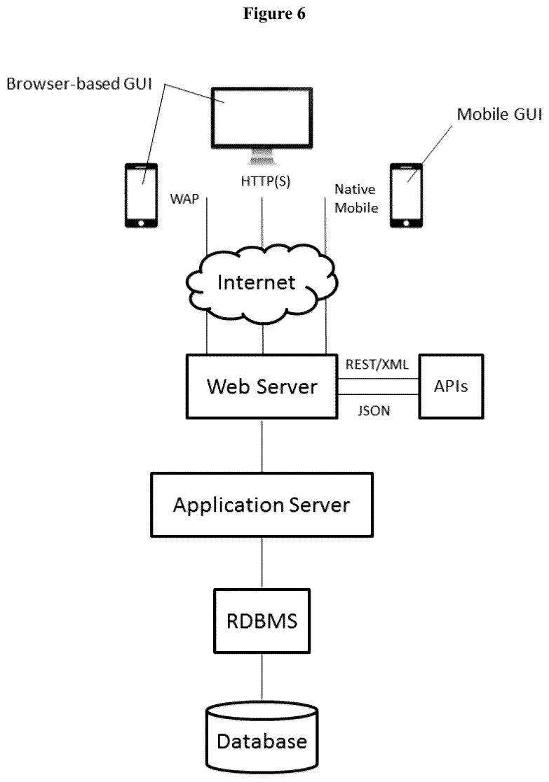

FIGS. 2A-4 show an exemplary graphical user interface (GUI) for implementing the methods herein. FIG. 2A shows a GUI for selecting a skin and hair side effect or a mood and stress side effect. Further, as shown, the GUI enables the subject to request further information regarding weight control, contraception, and digestion. FIG. 2B shows a GUI for learning about a skin and hair side effect. FIG. 3A shows a GUI for a subject to enter biological information. As shown therein, in some embodiments, the biological information is entered into the GUI by the subject through a health assessment, or can be received via a hormone test or a genetic test. FIG. 3B shows a GUI for a subject to order a test to submit a measured hormonal state. FIG. 4 shows a GUI for purchasing the test to submit the measured hormonal state.

### Example Process of Providing a Contraceptive Recommendation and Updating Relationships.

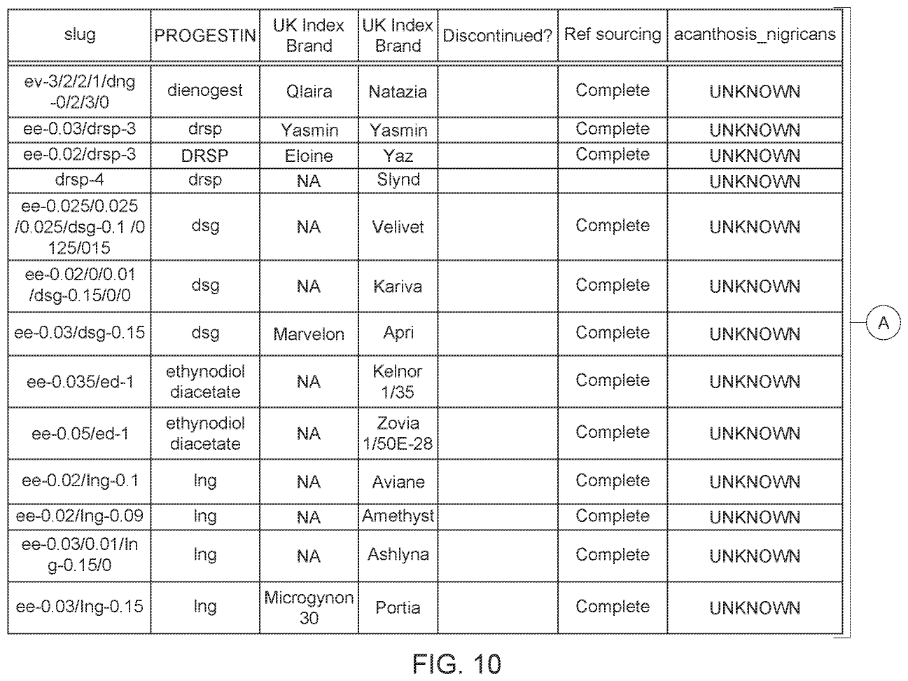

FIG. 8 depicts an example flow process for providing one or more contraceptive recommendations and updating one or more relationships. In this depicted example, server 120 and computing device 130 are in communication. In other embodiments, other processing devices may be used.

The process begins at step 802 with receiving an indication of one or more contraceptives. In some embodiments, the indication may be received from the computing device 130. In other embodiments, the indication may be received by a network. In yet another embodiment, the server 120 may determine the one or more contraceptives instead of receiving an indication.

At step 804, a recommendation is provided to computing device 130. In some embodiments, before providing the recommendation, the server 120 may determine one or more contraceptives to be indicated by the recommendation. The determination of the contraceptives may be based on relationships between side effects associated with the contraceptives and biological variables associated with the side effects. In particular, each side effect may be associated with a side effect severity, which may be related to one or more biological variables. The relationships may be used to determine the recommended one or more contraceptives using one or more techniques, such as those described with respect to FIG. 1B.

At step 806, the computing device 130 determines or more side effect factors. The one or more side effect factors may be determined based on information received about a user who has taken or who will take at least one of the recommended contraceptives. The information may be received by user input from the user. The information may include one or more images, such as images of the users face as described with respect to FIG. 1B.

At step 808, the computing device 130 provides the one or more side effect factors to the server 120.

At step 810, the server 120 updates one or more relationships based on the side effect factors. For example, the server 120 may further determine or update one or more side effect severities based on the one or more side effect factors, and those one or more side effect severities may be used to update the relationship between a corresponding side effect and a biological variable.

Thus, through process 800, the relationships may be used to provide recommendations to users, which may in turn result in side effect factors that may be used to further refine the relationships so that improved recommendations may be provided in the future.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### Computing System

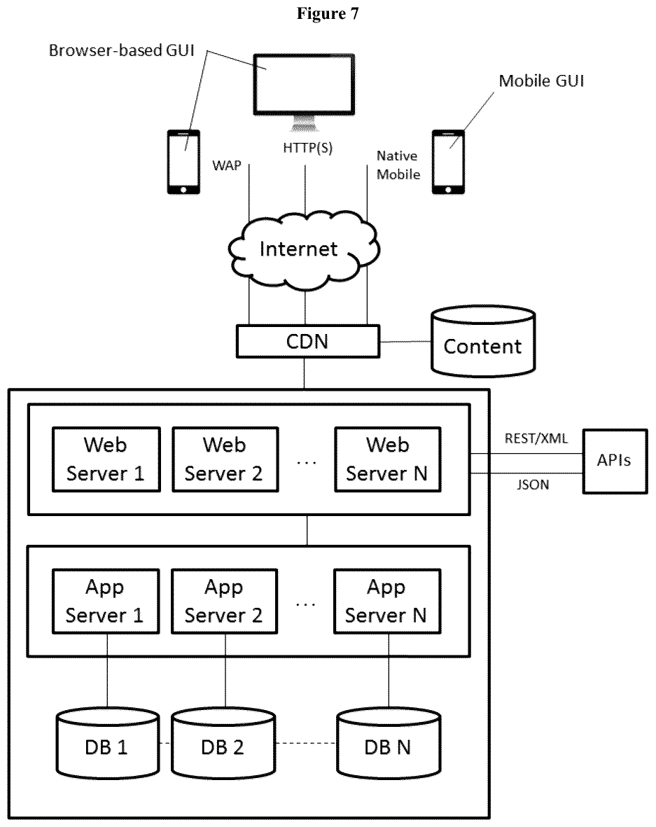

Referring to FIG. 5, a block diagram is shown depicting an exemplary machine that includes a computer system 500 (e.g., a processing or computing system) within which a set of instructions can execute for causing a device to perform or execute any one or more of the aspects and/or methodologies for static code scheduling of the present disclosure. The components in FIG. 5 are examples only and do not limit the scope of use or functionality of any hardware, software, embedded logic component, or a combination of two or more such components implementing particular embodiments.

Computer system 500 may include one or more processors 501, a memory 503, and a storage 508 that communicate with each other, and with other components, via a bus 540. The bus 540 may also link a display 532, one or more input devices 533 (which may, for example, include a keypad, a keyboard, a mouse, a stylus, etc.), one or more output devices 534, one or more storage devices 535, and various tangible storage media 536. All of these elements may interface directly or via one or more interfaces or adaptors to the bus 540. For instance, the various tangible storage media 536 can interface with the bus 540 via storage medium interface 526. Computer system 500 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 500 includes one or more processor(s) 501 (e.g., central processing units (CPUs) or general purpose graphics processing units (GPGPUs)) that carry out functions. Processor(s) 501 optionally contains a cache memory unit 502 for temporary local storage of instructions, data, or computer addresses. Processor(s) 501 are configured to assist in execution of computer readable instructions. Computer system 500 may provide functionality for the components depicted in FIG. 5 as a result of the processor(s) 501 executing non-transitory, processor-executable instructions embodied in one or more tangible computer-readable storage media, such as memory 503, storage 508, storage devices 535, and/or storage medium 536. The computer-readable media may store software that implements particular embodiments, and processor(s) 501 may execute the software. Memory 503 may read the software from one or more other computer-readable media (such as mass storage device(s) 535, 536) or from one or more other sources through a suitable interface, such as network interface 520. The software may cause processor(s) 501 to carry out one or more processes or one or more steps of one or more processes described or illustrated herein. Carrying out such processes or steps may include defining data structures stored in memory 503 and modifying the data structures as directed by the software.

The memory 503 may include various components (e.g., machine readable media) including, but not limited to, a random access memory component (e.g., RAM 504) (e.g., static RAM (SRAM), dynamic RAM (DRAM), ferroelectric random access memory (FRAM), phase-change random access memory (PRAM), etc.), a read-only memory component (e.g., ROM 505), and any combinations thereof. ROM 505 may act to communicate data and instructions unidirectionally to processor(s) 501, and RAM 504 may act to communicate data and instructions bidirectionally with processor(s) 501. ROM 505 and RAM 504 may include any suitable tangible computer-readable media described below. In one example, a basic input/output system 506 (BIOS), including basic routines that help to transfer information between elements within computer system 500, such as during start-up, may be stored in the memory 503.

Fixed storage 508 is connected bidirectionally to processor(s) 501, optionally through storage control unit 507. Fixed storage 508 provides additional data storage capacity and may also include any suitable tangible computer-readable media described herein. Storage 508 may be used to store operating system 509, executable(s) 510, data 511, applications 512 (application programs), and the like. Storage 508 can also include an optical disk drive, a solid-state memory device (e.g., flash-based systems), or a combination of any of the above. Information in storage 508 may, in appropriate cases, be incorporated as virtual memory in memory 503.

In one example, storage device(s) 535 may be removably interfaced with computer system 500 (e.g., via an external port connector (not shown)) via a storage device interface 525. Particularly, storage device(s) 535 and an associated machine-readable medium may provide non-volatile and/or volatile storage of machine-readable instructions, data structures, program modules, and/or other data for the computer system 500. In one example, software may reside, completely or partially, within a machine-readable medium on storage device(s) 535. In another example, software may reside, completely or partially, within processor(s) 501.

Bus 540 connects a wide variety of subsystems. Herein, reference to a bus may encompass one or more digital signal lines serving a common function, where appropriate. Bus 540 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 500 may also include an input device 533. In one example, a user of computer system 500 may enter commands and/or other information into computer system 500 via input device(s) 533. Examples of an input device(s) 533 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 533 may be interfaced to bus 540 via any of a variety of input interfaces 523 (e.g., input interface 523) including, but not limited to, serial, parallel, game port, USB, FIREWIRE, THUNDERBOLT, or any combination of the above.

In particular embodiments, when computer system 500 is connected to network 530, computer system 500 may communicate with other devices, specifically mobile devices and enterprise systems, distributed computing systems, cloud storage systems, cloud computing systems, and the like, connected to network 530. Communications to and from computer system 500 may be sent through network interface 520. For example, network interface 520 may receive incoming communications (such as requests or responses from other devices) in the form of one or more packets (such as Internet Protocol (IP) packets) from network 530, and computer system 500 may store the incoming communications in memory 503 for processing. Computer system 500 may similarly store outgoing communications (such as requests or responses to other devices) in the form of one or more packets in memory 503 and communicated to network 530 from network interface 520. Processor(s) 501 may access these communication packets stored in memory 503 for processing.

Examples of the network interface 520 include, but are not limited to, a network interface card, a modem, and any combination thereof. Examples of a network 530 or network segment 530 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 530, may employ a wired and/or a wireless mode of communication. In general, any network topology may be used.

Information and data can be displayed through a display 532. Examples of a display 532 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 532 can interface to the processor(s) 501, memory 503, and fixed storage 508, as well as other devices, such as input device(s) 533, via the bus 540. The display 532 is linked to the bus 540 via a video interface 522, and transport of data between the display 532 and the bus 540 can be controlled via the graphics control 521. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 532, computer system 500 may include one or more other peripheral output devices 534 including, but not limited to, an audio speaker, a printer, a storage device, and any combinations thereof. Such peripheral output devices may be connected to the bus 540 via an output interface 524. Examples of an output interface 524 include, but are not limited to, a serial port, a parallel connection, a USB port, a FIREWIRE port, a THUNDERBOLT port, and any combinations thereof.

In addition or as an alternative, computer system 500 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Sony® PS5®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

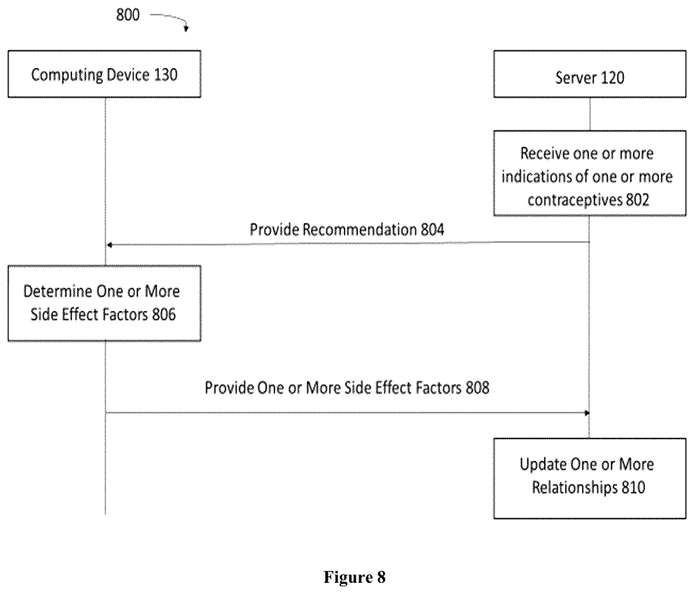

Referring to FIG. 6, in a particular embodiment, an application provision system comprises one or more databases 600 accessed by a relational database management system (RDBMS) 610. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 620 (such as Java servers, .NET servers, PHP servers, and the like) and one or more web servers 630 (such as Apache, IIS, GWS and the like). The web server(s) optionally expose one or more web services via app application programming interfaces (APIs) 640. Via a network, such as the Internet, the system provides browser-based and/or mobile native user interfaces.

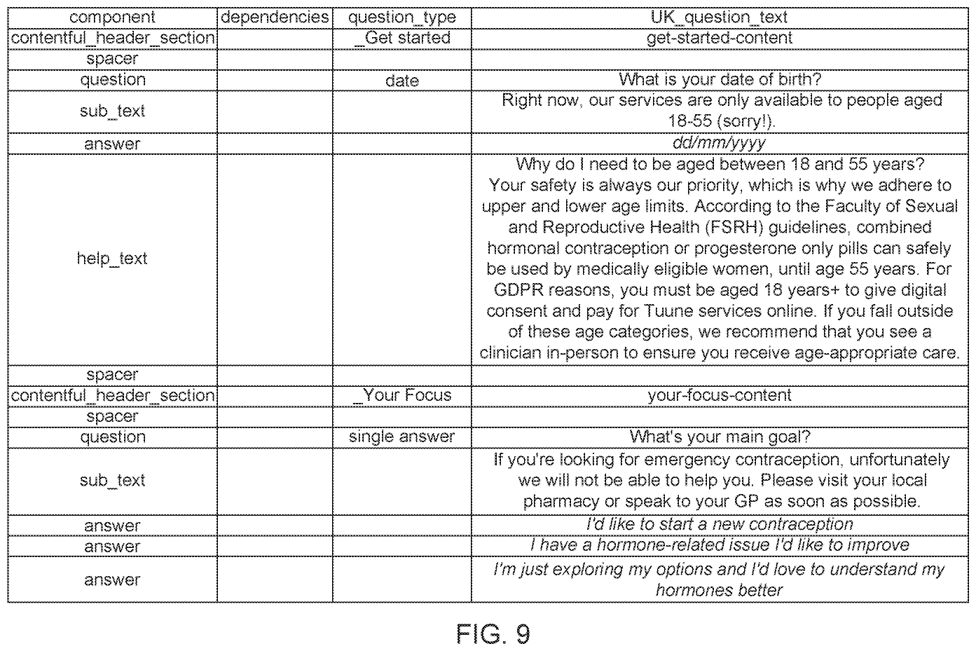

Referring to FIG. 7, in a particular embodiment, an application provision system alternatively has a distributed, cloud-based architecture 700 and comprises elastically load balanced, auto-scaling web server resources 710 and application server resources 720 as well synchronously replicated databases 730.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe Flash Player, Microsoft Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of the biological variables, facial metrics, and/or contraceptive scores described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Example Method for Providing an Optimal Contraceptive Recommendation

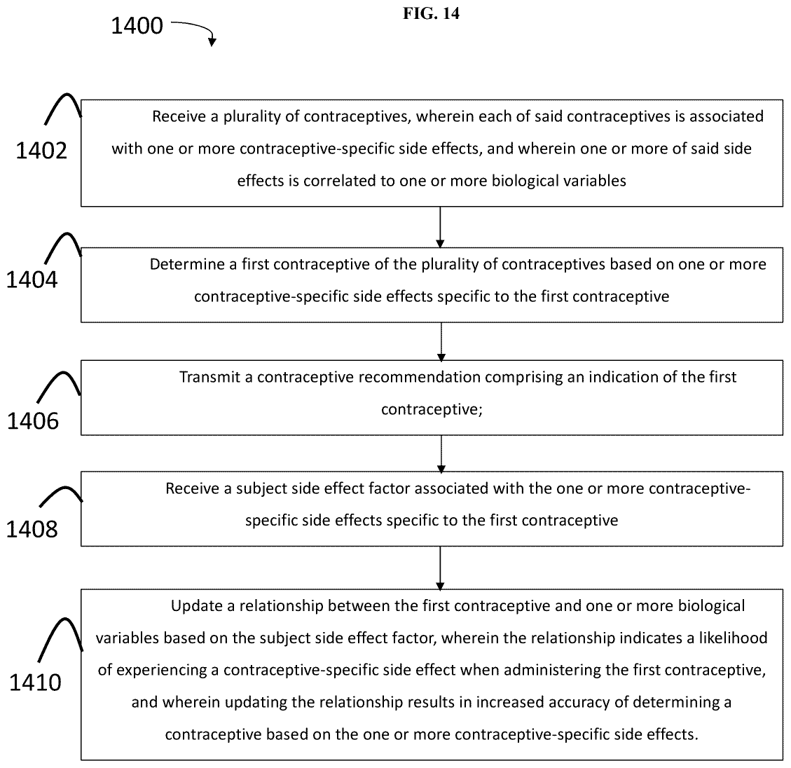

FIG. 14 depicts an example method for providing an optimal contraceptive recommendation based on relationships between contraceptive side effects and biological variables. In some embodiments, the method may be performed by a server (e.g., server 120 of FIG. 1). In other embodiments, the method may be performed by different processing device.

The method begins at step 1402 with receiving a plurality of contraceptives. In some embodiments, an indication of the plurality of contraceptives may be received instead of the plurality of contraceptives. In some embodiments, both the indication and the plurality of contraceptives may be received. Each contraceptive in the plurality of contraceptives may be associated with one or more contraceptive side effects. Each contraceptive side effect may further have an associated side effect severity. A plurality of relationships between the one or more side effects, one or more side effect severities, and/or one or more biological variables may be created. In some embodiments, a plurality of relationships between the one or more side effects, one or more side effect severities, and/or one or more biological variables that is specific to a single user may be created to generate a profile for that user.

At step 1404, a first contraceptive of the plurality of contraceptives is chosen based on the one or more side effects. In some embodiments, the first contraceptive is chosen based on the plurality of relationships. For example, for a specific user, one contraceptive may improve or worsen certain side effects, or make the side effect severity more or less severe, based on the biological variables associated with that user. Thus, certain techniques may be used, as described with respect to FIG. 1B, in order to choose the optimal contraceptive for the user.

At step 1406, a contraceptive recommendation may be transmitted to the user or a computing device associated with the user. The contraceptive recommendation may indicate a first contraceptive. In some embodiments, the contraceptive recommendation may indicate more than one contraceptive. In some embodiments, the more than one contraceptives may be ranked.

At step 1408, a subject side effect factor associated with the one or more contraceptive-specific side effects of the first contraceptive may be received. The subject side effect factor may include information input by the user. The subject side effect factor may also include one or more images of the user. The one or more images of the user may include images of the user's face. The side effect factor may be analyzed in order to determine one or more side effect severities or one or more biological variables associated with the user. The determined one or more side effect severities or one or more biological variables may be used to update the plurality of relationships, as described below.

At step 1410, at least one relationship of the plurality of relationships may be updated based on the side effect factor. Updating the at least one relationship of the plurality of relationships allows for more accurate contraceptive recommendations to be made because they updated relationships allow for more accurate predictions of how a user may respond to taking a contraceptive based on those biological variables. Thus, for each time that a user take a contraceptive, the relationships between the associated side effect and the biological variables may be updated to more accurately predict not only how the single user may respond, but also how the contraceptive generally affects other users, so the relationship between the side effect and the biological variable for the single user may be updated as well as the general relationship between the side effect of the contraceptive and the biological variable for all users may be updated. Therefore, with each update to a relationship of the plurality of relationships, future recommendations of contraceptive may be more accurately made.

### EXAMPLES

The following illustrative examples are representative of embodiments of the software applications, systems, and methods described herein and are not meant to be limiting in any way.

### Example 1

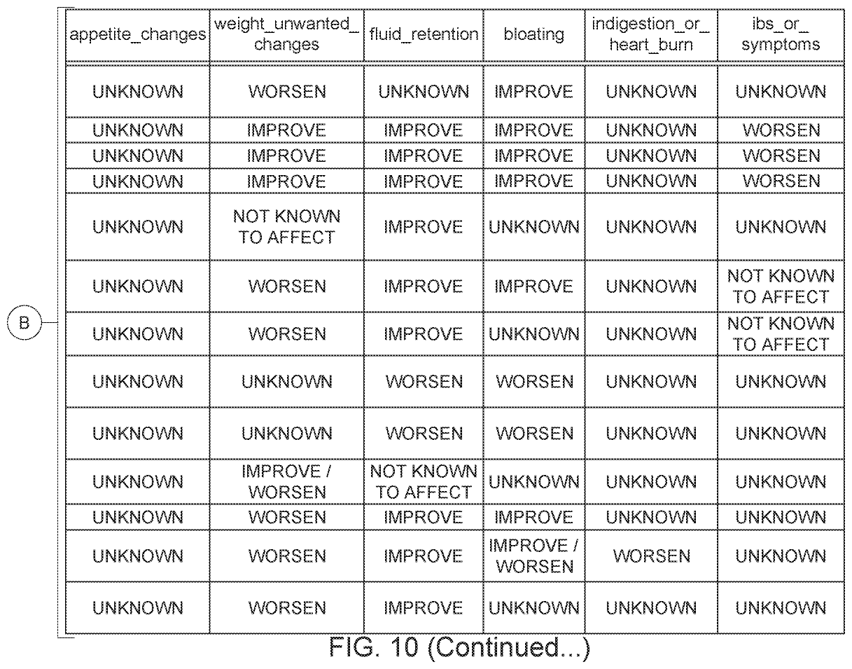

Over 3500 women were screened via a computer-implemented system. The women complete a detailed questionnaire of over 5850 questions, as partially displayed in FIG. 9, for assessing one or more of the biological variables described herein. FIG. 9 displays representative questions that were asked to the user (e.g., “What is your date of birth?” and “What's your main goal?”) as well as possible answers that may be selected through user input (e.g., a date of birth and at least one of “I'd like to start a new contraception”, “I have a hormone-related issue I'd like to improve”, and “I'm just exploring my options and I'd love to understand my hormones better”). In this particular example, a sub-text was also shown to the user to provide context to the questions that were asked and the answers that could be provided. The assessment of the one or more biological variables is compared to historical datasets concerning the one or more biological variables and their associations with hormonal states, along with academic research, is done by the algorithms described herein to generate hormonal states for the over 3500 women. Each hormonal state is further defined along an androgen axis and an estrogen axis. A personalized profile is created for each user including a weighted score for how severely each symptom affects the user.

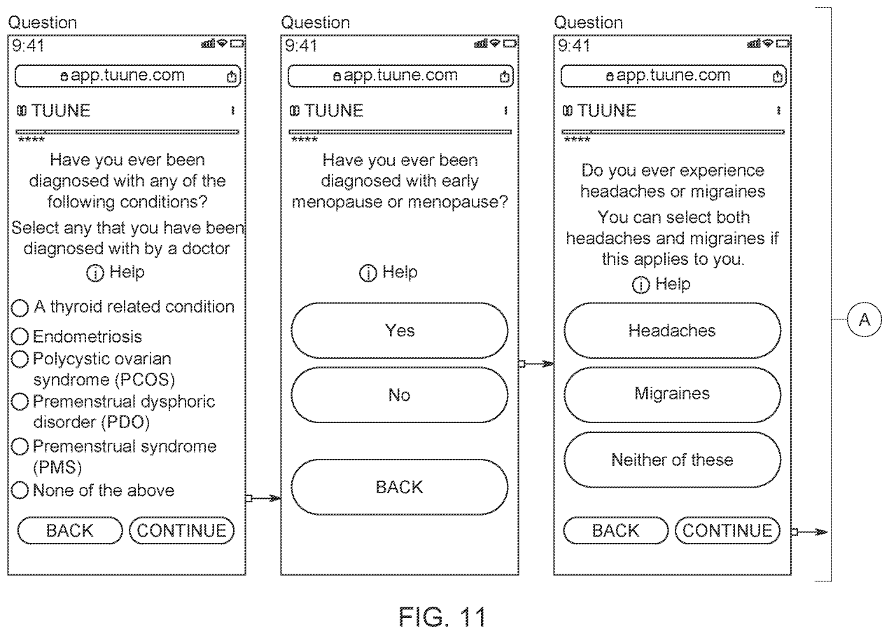

A matrix comprising 250 available contraceptives and known side effect symptoms is generated. A side effect severity score for each side effect symptom is generated for each of the 250 contraceptives, based upon historical datasets and academic research. This matrix is exemplified in Table 2, with a portion of the matrix depicted in FIG. 10. The side effect severity scores for each side effect symptom are summed for each of the 250 contraceptives.

TABLE 2

| | Side Effect | | | | | | | | |
|---|---|---|---|---|---|---|---|---|---|
| | | Break- | | | | | | Severe | |
| | | through | | | Breast | Weight | | menstrual | |
| Contraceptive | Acne | bleeding | Mood | Headaches | pain | gain | Libido | cramps | PMS |
| 1 | 2 | −1 | 2 | −2 | −1 | −3 | 2 | 2 | 3 |
| 2 | −3 | 2 | −3 | 2 | −3 | 0 | −3 | 3 | 0 |
| 3 | −1 | 0 | −3 | 0 | −1 | −1 | −3 | −2 | −3 |
| 4 | 3 | 3 | −1 | −1 | −1 | 2 | −1 | −3 | −1 |
| 5 | 0 | 2 | 2 | 2 | −1 | −1 | 2 | −1 | −1 |
| 6 | 2 | −1 | 1 | 1 | 2 | 0 | 1 | 2 | −1 |
| 7 | 0 | 3 | 2 | 2 | −1 | −3 | 2 | −3 | 3 |
| 8 | 2 | 2 | 0 | −2 | −1 | −1 | 0 | 3 | −3 |
| 9 | −3 | 2 | −1 | −2 | −1 | −3 | −1 | 2 | 0 |
| 10 | 3 | −3 | −1 | 2 | −3 | 2 | −1 | −1 | 0 |
| 11 | −1 | −2 | −2 | −2 | 2 | 0 | −2 | 0 | 3 |
| 12 | 2 | −1 | −3 | −2 | 3 | 0 | −3 | −1 | 2 |
| 13 | −1 | −3 | −3 | 2 | −2 | −2 | −3 | 2 | −1 |
| 14 | 2 | 3 | −1 | −1 | −3 | 0 | −1 | −2 | −3 |
| 15 | 2 | −2 | −1 | 2 | −3 | −1 | −1 | −1 | 3 |
| 16 | 0 | 2 | 0 | 3 | 2 | 3 | 0 | 3 | −1 |
| 17 | −3 | 2 | 0 | 0 | −2 | −1 | 0 | −1 | −3 |
| 18 | 3 | 0 | 0 | 2 | −1 | −3 | 0 | −2 | −1 |
| 19 | −2 | 3 | 0 | −1 | −3 | 0 | 0 | 3 | −3 |
| 20 | 3 | 3 | −1 | 3 | −3 | 3 | −1 | −2 | 2 |
| 21 | 3 | 0 | −1 | 2 | 0 | −2 | −1 | −2 | −2 |
| 22 | 2 | −3 | 3 | 2 | −3 | 0 | 3 | −3 | 2 |
| 23 | −1 | −2 | 2 | −1 | −2 | −3 | 2 | −2 | 3 |

The matrix as depicted in FIG. 10 further indicates contraceptives that were considered for recommendations as well as whether the contraceptive was known to worsen the side effect, improve the side effect, improve or worsen the side effect, or not effect the side effect, or whether the cause of the contraceptive to the side effect symptoms were unknown. Using the matrix, a profile for each woman could be generated, as described below.

The hormonal states of each of the 3500 women were correlated with the aggregate side effect severity scores for each of the contraceptives to generate correlation coefficients for each woman. Contraceptive recommendations were made for each of the 3500 women based on the correlation coefficient and historical datasets for each respective woman, which are included in each respective woman's profile. Upon generation of the contraceptive recommendation, each of the 3500 women indicate whether they desire the recommendation to be either: (1) sent to the woman with no additional recommendations; (2) passed along to a pharmaceutical distribution partner for allocation and delivery to the woman; or (3) passed along to a clinician for consultation with the woman.

Upon administration of the recommend contraceptive(s), some or all of the 3500 women provide feedback via a computer-implemented system, specifically on the onset of side effects. This feedback becomes a part of the historical datasets that are used to train the algorithm by machine learning to generate the hormonal state of the subject.

Utilizing the machine learning algorithm as described in the methods and systems described herein, subjects utilizing the systems and methods described herein experience a side effect burden of less than 52%.

### Example 2

50 women are given a computer-implemented system (e.g. mobile comprising a facial camera and uploaded with software, a laptop computer comprising a facial camera and uploaded with software) configured to identify facial metrics and changes thereof. Over the course of 1 day, 1 week, 2 week, and 1 month time periods, the women utilize the computer-implemented systems to identify their own facial metrics and changes thereof. The assessment of one or more facial metrics and changes thereof is compared to historical datasets concerning facial metrics and changes thereof and their associations with hormonal states, along with academic research, is done by the algorithms described herein to generate hormonal states for the 50 women. Each hormonal state is further defined along an androgen axis and an estrogen axis.

A matrix comprising 21 available contraceptives and known side effect symptoms is generated. A side effect severity score for each side effect symptom is generated for each of the 21 contraceptives, based upon historical datasets and academic research. This matrix is exemplified in Table 3. The side effect severity scores for each side effect symptom are summed for each of the 21 contraceptives.

TABLE 3

| | Side Effect | | | | | | | | |
|---|---|---|---|---|---|---|---|---|---|
| | | Break- | | | | | | Severe | |
| | | through | | | Breast | Weight | | menstrual | |
| Contraceptive | Acne | bleeding | Mood | Headaches | pain | gain | Libido | cramps | PMS |
| 1 | 2 | −1 | 2 | −2 | −1 | −3 | 2 | 2 | 3 |
| 2 | −3 | 2 | −3 | 2 | −3 | 0 | −3 | 3 | 0 |
| 3 | −1 | 0 | −3 | 0 | −1 | −1 | −3 | −2 | −3 |
| 4 | 3 | 3 | −1 | −1 | −1 | 2 | −1 | −3 | −1 |
| 5 | 0 | 2 | 2 | 2 | −1 | −1 | 2 | −1 | −1 |
| 6 | 2 | −1 | 1 | 1 | 2 | 0 | 1 | 2 | −1 |
| 7 | 0 | 3 | 2 | 2 | −1 | −3 | 2 | −3 | 3 |
| 8 | 2 | 2 | 0 | −2 | −1 | −1 | 0 | 3 | −3 |
| 9 | −3 | 2 | −1 | −2 | −1 | −3 | −1 | 2 | 0 |
| 10 | 3 | −3 | −1 | 2 | −3 | 2 | −1 | −1 | 0 |
| 11 | −1 | −2 | −2 | −2 | 2 | 0 | −2 | 0 | 3 |
| 12 | 2 | −1 | −3 | −2 | 3 | 0 | −3 | −1 | 2 |
| 13 | −1 | −3 | −3 | 2 | −2 | −2 | −3 | 2 | −1 |
| 14 | 2 | 3 | −1 | −1 | −3 | 0 | −1 | −2 | −3 |
| 15 | 2 | −2 | −1 | 2 | −3 | −1 | −1 | −1 | 3 |
| 16 | 0 | 2 | 0 | 3 | 2 | 3 | 0 | 3 | −1 |
| 17 | −3 | 2 | 0 | 0 | −2 | −1 | 0 | −1 | −3 |
| 18 | 3 | 0 | 0 | 2 | −1 | −3 | 0 | −2 | −1 |
| 19 | −2 | 3 | 0 | −1 | −3 | 0 | 0 | 3 | −3 |
| 20 | 3 | 3 | −1 | 3 | −3 | 3 | −1 | −2 | 2 |
| 21 | 3 | 0 | −1 | 2 | 0 | −2 | −1 | −2 | −2 |
| 22 | 2 | −3 | 3 | 2 | −3 | 0 | 3 | −3 | 2 |
| 23 | −1 | −2 | 2 | −1 | −2 | −3 | 2 | −2 | 3 |

The hormonal states of each of the 50 women are correlated with the aggregate side effect severity scores for each of the contraceptives to generate correlation coefficients for each woman. Contraceptive recommendations are made for each of the 50 women based on the correlation coefficient and historical datasets. Upon generation of the contraceptive recommendation, each of the 50 women indicate whether they desire the recommendation to be either: (1) sent to the woman with no additional recommendations; (2) passed along to a pharmaceutical distribution partner for allocation and delivery to the woman; or (3) passed along to a clinician for consultation with the woman.

Upon administration of the recommend contraceptive(s), some or all of the 50 women provide feedback via the computer-implemented system, specifically on the onset of side effects. This feedback becomes a part of the historical datasets that are used to train the algorithm by machine learning to generate the hormonal state of the subject.

Utilizing the machine learning algorithm as described in the methods and systems described herein, subjects utilizing the systems and methods described herein experience a side effect burden of less than 52%.

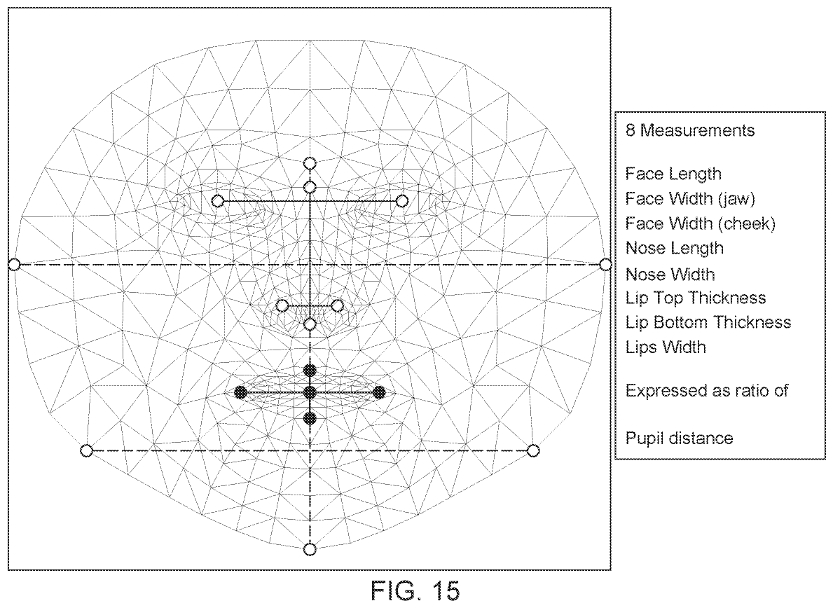

In a study of 4 women morning noon and evening photographs were taken with a camera phone every day for a period of one month. 8 measurements are taken from each photograph using a facial mesh with over 300 reference data points. The 8 measurements taken included Face Length, Face Width (jaw), Face Width (cheek), Nose Length, Nose Width, Lip Top Thickness, Lip Bottom Thickness, Lips Width (as shown in FIG. 15) and expressed as a ratio to pupil distance as an example of how to normalize the data. Two baseline periods are selected for each analysis, for analysis 1, period one correlated to days 1-8 of the menstrual cycle and one correlating to days 9-14. For analysis 2 period one correlated to days 1-12 of the menstrual cycle and period two correlating to the second half of the cycle namely days 13-28. The data from the 4 women are grouped and split into the baseline periods and compared. Using a classification model, each analysis performed better than the random-guess baseline although the difference was not significant.

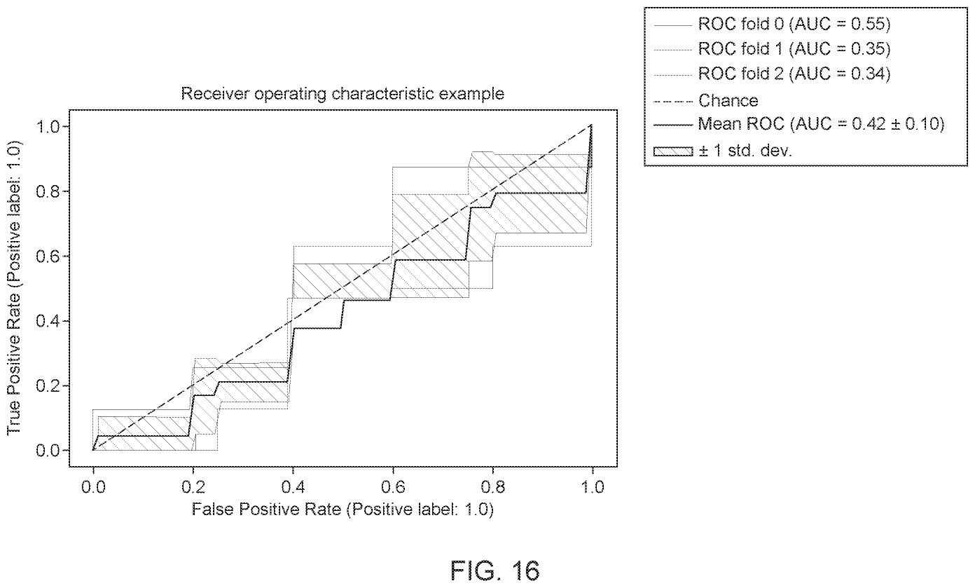

FIG. 16 is an example of the type of ROC curve seen and in this figure two classes compared were days 1-8, and days 9-14 to give ROC AUC 0.42+/−0.10. By expanding the date ranges to include more data, ROC analysis indicted that the most significant result was obtainable by including all of the data in a roughly even split (days 1-12 and 13-28), giving a mean ROC AUC of 0.61+/−0.14.

It is clear that with more participants, taking more photos, over a longer period of time and using more metrics and combinations of facial metrics and normalizations that we will be able to improve on the AUC and statistical significance of the data to a point where individual photos from the same individual can be used to determine a hormonal state of that individual.

These results indicate that hormonal profiles are drastically different in the first half of the menstrual cycle compared to the second half. Further, this data shows that if facial metrics change in the same two periods of a menstrual cycle, then there will be a correlation between the change in facial metrics and hormone levels (e.g. the “hormonal state”) of an individual.

### Example 3

During initial onboarding, Karen provides the biological information through a health assessment regarding her age of 48 and history of breast cancer. Side effect severities are calculated, per Table 4 below, based on her biological information for each of drugs A-E, and side effects a, b, and c. As each of the potential side effects for drug D has a high correlation coefficient with a history of breast cancer, drug D is associated with a high total side effect severity and Karen is alerted not to take drug D as a contraceptive. Instead, Karen is provided with drugs A, B, and E as contraceptive medication. Two months after taking drug E, however, Karen provides a subject side effect factor that the severity of side effect b was greater than expected. As such, per Table 4 below, the correlation coefficient between breast cancer and side effect b is increased to improve determination of contraceptive recommendations over time.

TABLE 4

Side Effect

| Drug | a | b | c | Total |
|---|---|---|---|---|
| A | 1 | 5 | 2 | 8 |
| B | 3 | 2 | 3 | 8 |
| C | 2 | 4 | 5 | 11 |
| D | 5 | 5 | 5 | 15 |
| E | 4 | 1 | 2 | 7 |

### Example 4

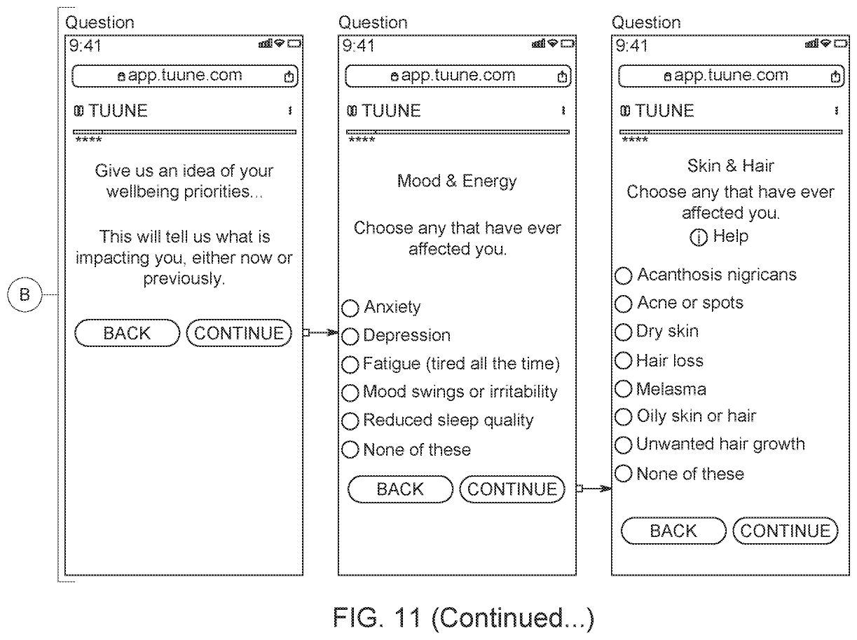

An example user interface is displayed to a woman seeking a contraceptive recommendation as depicted in FIG. 11. The user interface has multiple screens, with many screens displaying a new question for the woman to answer. The woman answers by providing user input. The user interface may receive user input through a touch screen, electronic input, or speaker input. For some questions, the woman can select multiple answers. For other questions, the woman can select only one answer.

### Example 5

Four women underwent the process as described in Example 1. Each of the four women received a contraceptive recommendation and took the respectively recommended contraceptive. Each of the four women, who had been each been taking a different contraceptive before receiving their respectively recommended contraceptive, recorded improvements or deteriorations regarding symptoms of one or more side effects and provided indications of the improvements or deteriorations through user input or facial imaging (e.g., side effect factors). The relationships between the side effect severities and the biological variables for the four women were updated. Two of the four women underwent the process again and received a new contraceptive recommendations based on the updated relationships. The two women who underwent the process again recorded improvements or deteriorations regarding symptoms of one or more side effects based on the change of the contraceptive and provided indications of those improvements or deteriorations through user input or facial imaging, which were used to further update the relationships between the side effect severities and biological variables for the two women.

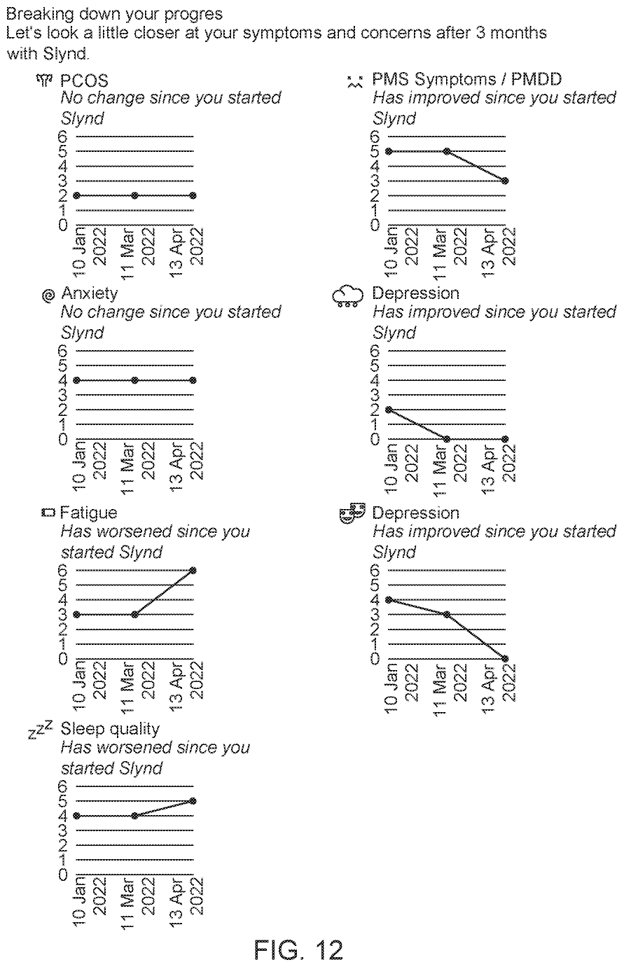

FIG. 12 illustrates the improvements or deteriorations regarding symptoms for one of the two women who switched between the three contraceptives. For example, the anxiety of the woman did not change when she switched between the contraceptives. However, the fatigue of the woman worsened between the second contraceptive and the third contraceptive even though it did not change from the first contraceptive to the second contraceptive. Further, the depression of the woman improved from the first contraceptive to the second contraceptive, but not from the second contraceptive to the third contraceptive.

### Example 6

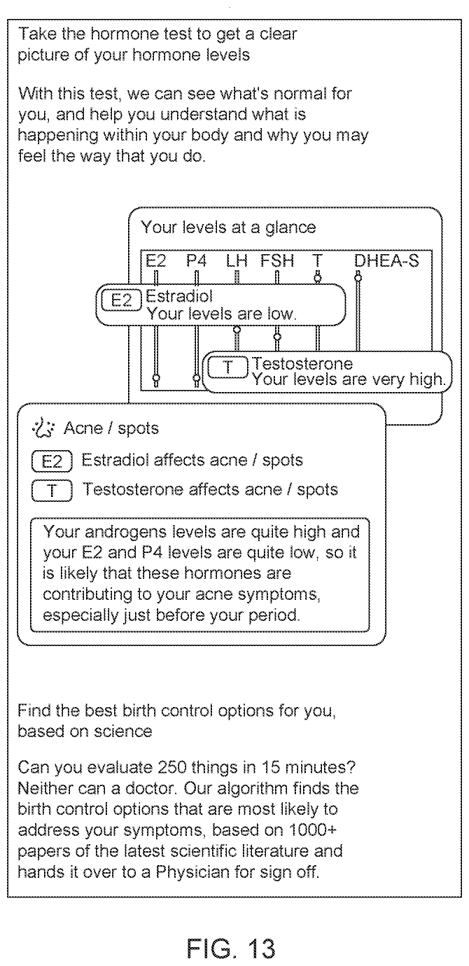

A number of women underwent the process as described in Example 1. A hormone test was carried out to confirm that the symptoms of side effects were hormone related rather than genetic in order to confirm that the optimal contraceptive was recommended. The hormone test results were provided to the woman along with explanations of relationships between hormone levels and symptoms, as depicted in FIG. 13.

### Example 7

A number of women underwent the processes as described in Examples 1 and 6, which identified, in this example, (Peri)-menopause. (Peri)-menopause was identified based on a respective hormonal state for each of the number of women. The hormonal state was determined based on the received side effect factors from the number of women indicating the side effects, as well as hormone test results permitting another hormone therapy to be prescribed along with or instead of the hormonal contraception.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

1.-84. (canceled)

85. A computer-implemented method for determining a contraceptive recommendation for a subject, said method comprising:
(a) receiving a plurality of contraceptives, wherein each contraceptive of said plurality of contraceptives is associated with one or more contraceptive-specific side effects, and wherein one or more of said one or more contraceptive-specific side effects is correlated to one or more biological variables;
(b) determining a first contraceptive of the plurality of contraceptives based on one or more contraceptive-specific side effects specific to the first contraceptive;
(c) determining a contraceptive recommendation comprising an indication of the first contraceptive using a contraceptive machine learning algorithm;
(d) transmitting said contraceptive recommendation;
(e) receiving a subject side effect factor associated with the one or more contraceptive-specific side effects specific to the first contraceptive; and
(f) updating a relationship between the first contraceptive and one or more biological variables based on the subject side effect factor, wherein the relationship indicates a likelihood of experiencing a contraceptive-specific side effect when administering the first contraceptive, and wherein updating the relationship results in increased accuracy of determining a contraceptive based on the one or more contraceptive-specific side effects.

86. The method of claim 1, wherein each of said one or more contraceptive-specific side effects comprises a side effect severity and the first contraceptive-specific side effect comprises a first side effect severity.

87. The method of claim 2, wherein the relationship between the first contraceptive and one or more biological variables comprises the first side effect severity correlating to one or more biological variables through a first correlation coefficient.

88. The method of claim 2, wherein determining the first contraceptive of the plurality of contraceptives based on one or more contraceptive-specific side effects specific to the first contraceptive comprises:
comparing side effect severities associated with the contraceptive-specific side effects specific to the first contraceptive and side effect severities associated with one or more contraceptive-specific side effects specific to a second contraceptive; and
determining the first contraceptive based on the comparing.

89. The method of claim 85, wherein said one or more biological variables comprise a hormonal state of said subject.

90. The method of claim 89, wherein said hormonal state comprises quantification on an androgen axis, an estrogen axis, or both.

91. The method of any one of claim 89, wherein said hormonal state is determined by:
(a) receiving one or more images of the face of said subject;
(b) measuring one or more facial metrics based on said one or more images;
(c) generating a subject-specific score from said one or more facial metrics;
(d) receiving one or more verified hormonal states, each of said verified hormonal states associated with a verified hormonal state;
(e) determining said hormone level of said subject based on said one or more verified hormonal states and said subject-specific score; and
(f) providing an output representing said hormonal state of said subject.

92. The method of claim 91, wherein said one or more facial metrics comprise a facial width, facial height, facial coloration, mandibular width, mandibular contour, nasal width, or any combination thereof.

93. The method of claim 91, wherein (c) is performed by a facial metric machine learning algorithm, wherein said facial metric machine learning algorithm is trained by a method comprising:
(a) collecting two or more sets of facial metrics from a database, wherein each set of said two or more sets of facial metrics is associated with a validated subject-specific score;
(b) creating a first training set comprising said collected set of said two or more sets of facial metrics;
(c) training a neural network in a first stage to determine said subject-specific score using said first training set;
(d) creating a second training set comprising said first training set and said two or more sets of facial metrics whose subject-specific score was determined beyond a set threshold from said validated subject-specific score; and
(e) training said machine learning algorithm in a second stage using said second training set.

94. The method of claim 85, further comprising receiving a biological variable value for one or more of the biological variables associated with said subject.

95. The method of claim 85, wherein said contraceptive machine learning algorithm is trained by a method comprising:
(a) receiving two or more of said contraceptive-specific side effects from a database, wherein each of said two or more contraceptive-specific side effects is associated with said one or more of said biological variables by a verified biological correlation coefficient;
(b) creating a first training set comprising said two or more contraceptive-specific side effects;
(c) training a neural network in a first stage to determine a biological correlation coefficient;
(d) creating a second training set comprising said first training set and said contraceptive-specific side effects of said two or more said contraceptive-specific side effects whose biological correlation coefficient was determined to be different than said verified biological correlation coefficient by a set value; and
(e) training said machine learning algorithm in a second stage using said second training set.

96. The method of any one of claim 85, wherein determining said contraceptive recommendation is determined based on an average of side effect severities of each of said one or more contraceptive-specific side effects, a sum of said side effect severities of each of said one or more contraceptive-specific side effects, or both.

97. A system, comprising:
one or more processors; and
a memory comprising executable instructions which, when executed by the one or more processors, cause the system to:
(a) receive a plurality of contraceptives, wherein each contraceptive of said plurality of contraceptives is associated with one or more contraceptive-specific side effects, and wherein one or more of said one or more contraceptive-specific side effects is correlated to one or more biological variables;
(b) determine a first contraceptive of the plurality of contraceptives based on one or more contraceptive-specific side effects specific to the first contraceptive;
(c) determine a contraceptive recommendation comprising an indication of the first contraceptive using a contraceptive machine learning algorithm;
(d) transmit said contraceptive recommendation;
(e) receive a subject side effect factor associated with the one or more contraceptive-specific side effects specific to the first contraceptive; and
(f) update a relationship between the first contraceptive and one or more biological variables based on the subject side effect factor, wherein the relationship indicates a likelihood of experiencing a contraceptive-specific side effect when administering the first contraceptive, and wherein updating the relationship results in increased accuracy of determining a contraceptive based on the one or more contraceptive-specific side effects.

98. The system of claim 97, wherein each of said one or more contraceptive-specific side effects comprises a side effect severity and the first contraceptive-specific side effect comprises a first side effect severity.

99. The system of claim 98, wherein the relationship between the first contraceptive and one or more biological variables comprises the first side effect severity correlating to one or more biological variables through a first correlation coefficient.

100. The system of claim 98, wherein determining a first contraceptive of the plurality of contraceptives based on one or more contraceptive-specific side effects specific to the first contraceptive comprises:
comparing side effect severities associated with the contraceptive-specific side effects specific to the first contraceptive and side effect severities associated with one or more contraceptive-specific side effects specific to a second contraceptive; and
determining the first contraceptive based on the comparing.

101. The system of claim 97, wherein said one or more biological variables comprise a hormonal state of said subject.

102. A non-transitory, computer-readable medium comprising executable instructions, wherein when a processor, when executing the executable instructions, performs a method for determining a contraceptive recommendation for a subject, the method comprising:
(a) receiving a plurality of contraceptives, wherein each contraceptive of said plurality of contraceptives is associated with one or more contraceptive-specific side effects, and wherein one or more of said one or more contraceptive-specific side effects is correlated to one or more biological variables;
(b) determining a first contraceptive of the plurality of contraceptives based on one or more contraceptive-specific side effects specific to the first contraceptive;
(c) determining a contraceptive recommendation comprising an indication of the first contraceptive using a contraceptive machine learning algorithm;
(d) transmitting said contraceptive recommendation;
(e) receiving a subject side effect factor associated with the one or more contraceptive-specific side effects specific to the first contraceptive; and
(f) updating a relationship between the first contraceptive and one or more biological variables based on the subject side effect factor, wherein the relationship indicates a likelihood of experiencing a contraceptive-specific side effect when administering the first contraceptive, and wherein updating the relationship results in increased accuracy of determining a contraceptive based on the one or more contraceptive-specific side effects.

103. The non-transitory, computer-readable medium of claim 102, wherein each of said one or more contraceptive-specific side effects comprises a side effect severity and the first contraceptive-specific side effect comprises a first side effect severity.

104. The non-transitory, computer-readable medium of claim 102, wherein the relationship between the first contraceptive and one or more biological variables comprises the first side effect severity correlating to one or more biological variables through a first correlation coefficient.
